SEQUENCE LISTING (13 sequences)

SEQ ID NO: 1 - DNA, 1095 bp, Homo sapiens
atgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 2 - protein, 365 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 3 - DNA, 2440 bp, Homo sapiens
cggccttgtcagctcacagcaggcgttaacagcctctaattgaggaaactgtggctggacaggttgcaaggcagttctgctccccatcgtcctcttgctgactggggactgctgagcccgtgcacggcagagagtctggtggggtggaggggctggcctggcccctctgtcctgtggaaatgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcctagttgctgcagcaattctcacctttcttgcacatcagcatctgctttgggaattggcacagtggatgacggcacaggagtctctatagaacacttcctagtctggagaggatatggaaatttgttcttgttctatattttgttttgaaaatgatgtctaacaaccatgataagagcaaggctgttaaataatatcttccaatttacagatcagacatgaatgggtggaggggttaggttgttcacaaaaggccacattccaagtatttgtaatctagaaagtgttatgtaagtgatgttattagcatcgagattccctccacctgattttcaagctgtcacttgtttccttttctcccctctctgggttgactgcatttctagactctcgccggcccaggcccatcttccaaagcaagaggaaggaatgataatggtgactcaggggaagaagaaacagccctcctctgaaagcctggactgtccggctgtgaactggctggcaggttctgcacgtgggtgggggccagggcctgggctttactcaattgcagagaaaaaactttctccctgcatctcatacctttacctctggccagttggccaccagggggagtgggctgaagggagagtagatggtgcaaagcaagcccatctctgagtagaaaaatcacccagagcacatgctgacctgataactggggtgttgagaccagctttgtccatggtatgatgtttgatttatgaagacgcattgttagaaatccatttggcttcttcatagaagtggcttcccagaggaagaggcctctcagaaaccatgttctatttaagttctgagtcctgatgagtgttccccaggatgcacattgaagggagggctcaggcagctgagggctgagaatgaggcagttggaatctagacactatgctgggttccctgagtcgtcaggccagacatttcaacaaggctgtggggagcagggctgtgactctggctgagcccaggaaagcgacaagggtgaactgggagaggacttactcagagaccccaacaggtgatactgcacaaagcctggttcttcaattttcctaccctgtatctaacataggagtttcatataaaacggtgatatcatgcagatgcagtctgaattccttgcctg

SEQ ID NO: 4 - protein, 398 aa, Mus musculus
MLGQAVLFTTFLLLRAHQGQDCPDSSEEVVGVSGKPVQLRPSNIQTKDVSVQWKKTEQGSHRKIEILNWYNDGPSWSNVSFSDIYGFDYGDFALSIKSAKLQDSGHYLLEITNTGGKVCNKNFQLLILDHVETPNLKAQWKPWTNGTCQLFLSCLVTKDDNVSYAFWYRGSTLISNQRNSTHWENQIDASSLHTYTCNVSNRASWANHTLNFTHGCQSVPSNFRFLPFGVIIVILVTLFLGAIICFCVWTKKRKQLQFSPKEPLTIYEYVKDSRASRDQQGCSRASGSPSAVQEDGRGQRELDRRVSEVLEQLPQQTFPGDRGTMYSMIQCKPSDSTSQEKCTVYSVVQPSRKSGSKKRNQNYSLSCTVYEEVGNPWLKAHNPARLSRRELENFDVYS

SEQ ID NO: 5 - DNA, 1147 bp, Mus musculus
atgttggggcaagctgtcctgttcacaaccttcctgctcctcagggctcatcagggccaagactgcccagattcttctgaagaagtggttggtgtctcaggaaagcctgtccagctgaggccttccaacatacagacaaaagatgtttctgttcaatggaagaagacagaacagggctcacacagaaaaattgagatcctgaattggtataatgatggtcccagttggtcaaatgtatcttttagtgatatctatggttttgattatggggattttgctcttagtatcaagtcagctaagctgcaagacagtggtcactacctgctggagatcaccaacacaggcggaaaagtgtgcaataagaacttccagcttcttatacttgatcatgttgagacccctaacctgaaggcccagtggaagccctggactaatgggacttgtcaactgtttttgtcctgcttggtgaccaaggatgacaatgtgagctacgccttttggtacagagggagcactctgatctccaatcaaaggaatagtacccactgggagaaccagattgacgccagcagcctgcacacatacacctgcaacgttagcaacagagccagctgggcaaaccacaccctgaacttcacccatggctgtcaaagtgtcccttcgaatttcagatttctgccctttggggtgatcatcgtgattctagttacattatttctcggggccatcatttgtttctgtgtgtggactaagaagaggaagcagttacagttcagccctaaggaacctttgacaatatatgaatatgtcaaggactcacgagccagcagggatcaacaagggacaaagagaattggacaggcgtgtttctgaggtgctggagcagttgccacagcagactttccctggagatagaggcaccatgtactctatgatacagtgcaagccttctgattccacatcacaagaaaaatgtacagtatattcagtagtccagccttccaggaagtctggatccaagaagaggaaccagaactattccttaagttgtaccgtgtacgaggaggttggaaacccatggctcaaagctcacaaccctgccaggctgagccgcagagagctggagaactttgatgtctactcctag

SEQ ID NO: 6 - protein, 451 aa, Artificial Sequence [sequence omitted]

SEQ ID NO: 7 - protein, 243 aa, Artificial Sequence
  Synthetic
  PEPTIDE (1)..(221): human NAIL sequences
  PEPTIDE (222)..(226): spacer
  PEPTIDE (227)..(234): Flag tag
  PEPTIDE (235)..(237): spacer
  PEPTIDE (238)..(243): polyhistidine tag
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRRSGSSDYKDDDDKGSSHHHHHH

SEQ ID NO: 8 - protein, 272 aa, Artificial Sequence
  Synthetic
  PEPTIDE (1)..(221): human NAIL sequences
  PEPTIDE (222)..(226): spacer
  PEPTIDE (227)..(264): leucine zipper tag
  PEPTIDE (265)..(266): spacer
  PEPTIDE (267)..(272)
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRRSGSSRMKQIEDKIEEILSKIYHIENEIARIKKLIGERGTSSRGSHHHHHH

SEQ ID NO: 9 - DNA, 1098 bp, Homo sapiens
atgctggggcaagtggtcaccctcatactcctcctgctcctcaaggtgtatcagggcaaaggatgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttccaggtttttgtatttgataaagttgagaaaccccgcctacaggggcaggggaagatcctggacagagggagatgccaagtggctctgtcttgcttggtctccagggatggcaatgtgtcctatgcttggtacagagggagcaagctgatccagacagcagggaacctcacctacctggacgaggaggttgacattaatggcactcacacatatacctgcaatgtcagcaatcctgttagctgggaaagccacaccctgaatctcactcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcctag

SEQ ID NO: 10 - protein, 243 aa, Homo sapiens [sequence omitted]

SEQ ID NO: 11 - protein, 8 aa, Artificial Sequence
  Synthetic
DYKDDDDK

SEQ ID NO: 12 - protein, 27 aa, Artificial Sequence
  Synthetic
PDVASLRQQVEALQGQVQHLQAAFSQY

SEQ ID NO: 13 - protein, 33 aa, Artificial Sequence
  Synthetic
RMKQIEDKIEEILSKIYHIENEIARIKKLIGER

(19) United States
(12) Patent Application Publication
(10) Pub. No.: US 2008/0081043 A1
(43) Pub. Date: Apr. 3, 2008

(54) NK cell activation inducing ligand (NAIL) DNA and polypeptides, and use thereof

(76) Inventors: Marek Z. Kubin, Bainbridge Island, WA (US); Raymond G. Goodwin, Seattle, WA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/824,835

(22) Filed: Jul. 3, 2007

Related U.S. Application Data
(63) Continuation of application No. 09/667,859, filed on Sep. 20, 2000.
Application No. 09/667,859 is a continuation of application No. PCT/US99/06215, filed on Mar. 23, 1999.
(60) Provisional application No. 60/079,845, filed on Mar. 27, 1998.
(60) Provisional application No. 60/096,750, filed on Aug. 17, 1998.

(51) Int. Cl. A61K 39/00; A61P 43/00; C07K 14/00; C07K 16/18; C12N 15/00; C12N 15/11; C12N 5/06; C12P 21/04; G01N 33/00; G01N 33/566

(52) U.S. Cl. 424/185.1; 435/320.1; 435/325; 435/375; 435/69.1; 435/7.21; 436/86; 530/350; 530/387.9; 530/388.1; 536/23.1

(57) ABSTRACT

The invention is directed to purified and isolated novel NAIL polypeptides, the nucleic acids encoding such polypeptides, processes for production of recombinant forms of such polypeptides, antibodies generated against these polypeptides, fragmented peptides derived from these polypeptides, and the uses of the above.

### CROSS REFERENCE TO RELATED APPLICATIONS

This application claims the benefit of U.S. Provisional Application Ser. No. 60/079,845, filed Mar. 27, 1998, and U.S. Provisional Application Ser. No. 60/096,750, filed Aug. 17, 1998, both of which are specifically incorporated herein by reference.

### BACKGROUND OF THE INVENTION

1. Field of the Invention

The invention is directed to isolated and purified NK Cell Activation Inducing Ligand (NAIL) polypeptides, the nucleic acids encoding such polypeptides, processes for production of recombinant forms of such polypeptides, antibodies generated against these polypeptides, peptides derived from these polypeptides, and the use of these nucleic acid molecules, polypeptides and antibodies directed against these polypeptides as modulators of natural killer cell, cytotoxic T cell, and B cell activity, for the selective enrichment of specific cell populations, for inducing cytokine production and release, and for detecting and inhibiting NAIL's binding to its counter-structure, CD48.

2. Background

Natural Killer Cells

One of the major types of circulating mononuclear cells is that of the natural killer, or NK, cell (M. Manoussaka et al., Journal of Immunology 158:112-119, 1997). Originally defined based on their ability to kill certain tumors and virus-infected cells, NK cells are now known as one of the components of the early, innate immune system. In addition to their cytotoxic capabilities, NK cells serve as regulators of the immune response by releasing a variety of cytokines. In addition, the generation of complex immune responses is facilitated by the direct interaction of NK cells with other cells via various surface molecules expressed on the NK cells.

NK cells are derived from bone marrow precursors (O. Haller et al., Journal of Experimental Medicine 145:1411-1420, 1977). NK cells appear to be closely related to T cells, and the two cell types share many cell surface markers (M. Manoussaka et al., 1997). As noted above, these cell surface markers play a significant role in NK cell activity. For example, murine NK cells express specific antigens on their surfaces, such as asialo GM1, NK1, and NK2 antigens (D. See et al., Scand. J. Immunol. 46:217-224, 1997), and the administration of antibodies against these antigens results in depletion of NK cells in vivo (Id.). More significantly, the depletion of NK cells can result in a decreased resistance to target tissue infection by viruses (Id.). In addition, in earlier studies, antibodies directed against CD2 and CD11a inhibit the cytotoxic effect of NK cells (O. Ramos et al., J. Immunol. 142:4100-4104, 1989; C. Scott et al., J. Immunol. 142:4105-4112, 1989.

Similarly to cytotoxic T lymphocytes (CTL), NK cells exert a cytotoxic effect by lysing a variety of cell types (G. Trinichieri, 1989). These include normal stem cells, infected cells, and transformed cells (D. See et al., Scand. J. Immunol. 46:217-224, 1997). The lysis of cells occurs through the action of cytoplasmic granules containing proteases, nucleases, and perforin (D. See et al., 1997). Cells that lack MHC class I are also susceptible to NK cell-mediated lysis (H. Reyburn et al., Immunol. Rev. 155:119-125, 1997). In addition, NK cells exert cytotoxicity in a non-MHC restricted fashion (E. Ciccione et al., J. Exp. Med. 172:47, 1990; A. Moretta et al., J. Exp. Med. 172:1589, 1990; and E. Ciccione et al., J. Exp. Med. 175:709). NK cells can also lyse cells by antibody-dependent cellular cytotoxicity (D. See et al., 1997).

As noted above, NK cells mediate some of their functions through the secretion of cytokines, such as interferon γ (IFN-γ), granulocyte-macrophage colony-stimulating factors (GM-CSFs), tumor necrosis factor α (TNF-α), macrophage colony-stimulating factor (M-CSF), interleukin-3 (IL-3), and IL-8 (P. Scott and G. Trinichieri, 1995).

In addition, cytokines can influence NK behavior. For example, cytokines including IL-2, IL-12, TNF-α, and IL-1 can induce NK cells to produce cytokines (P. Scott and G. Trinichieri, 1995). IFN-α and IL-2 are strong inducers of NK cell cytotoxic activity (G. Trinichieri et al., Journal of Experimental Medicine 160:1147-1169, 1984; G. Trinichieri and D. Santoli, Journal of Experimental Medicine 147: 1314-1333, 1977). The presence of IL-2 both stimulates and expands NK cells (K. Oshimi, International Journal of Hematology 63:279-290, 1996). IL-12 has been shown to induce cytokine production from T and NK cells, and augment NK cell-mediated cytotoxicity (M. Kobayashi et al., Journal of Experimental Medicine 170:827-846, 1989). Other molecules have been shown to suppress the activation of NK cells (G. Gatti et al., Brain Behav. Immun. 7:16-28, 1993; M. De Martino et al., Clin. Exp. Immunol. 61:90-95, 1985; L. Pricop et al., Immunology 151:3018-3129, 1993).

As cytotoxic agents, NK cells have been shown to destroy both extracellular protozoa and the cells infected by protozoa (T. Scharton-Kersten and A. Sher, Current Opinion in Immunology 9:44-51, 1997). In most instances, cytotoxic activity appears to be dependent upon lymphokine activation (T. Scharton-Kersten and A. Sher, 1997). The activation of NK cells by protozoa is thought to involve an indirect (cytokine-mediated) mechanism involving the production of IL-12 and TNF-α (T. Scharton-Kersten and A. Sher, 1997). IL-10 and TGF-β have been shown to inhibit IFN-γ production and cytotoxicity of NK cells, suggesting that the activity of NK cells is tightly regulated (T. Scharton-Kersten and A. Sher, 1997).

In addition, NK cells are important in the early defense against many viral infections. Indeed, NK cells have been implicated as mediators of host defenses against infection in humans with varicella zoster, herpes simplex, cytomegalovirus, Epstein-Barr virus, hepatitis B, and hepatitis C viruses (D. See et al., 1997). Many viruses induce NK cell cytotoxicity, including herpesvirus and cytomegalovirus (C. Biron, Current Opinion in Immunology 9:24-34, 1997). In general, viral infection induces IFN-α/β which thereafter induce NK cell activity (C. Biron, 1997). The NK1+CD3− population of NK cells is the subset activated by viral infection (C. Biron, 1997).

As with protozoan infection, the response of NK cells to viral infection involves direct cytotoxicity and production of various cytokines such as IFN-γ and TNF-α, and is regulated by cytokines such as IL-12, IL-1, IFN-α/β, IL-15, TGF-β, and IL-10 produced by other cells during viral infection (C. Biron, 1997). Most of these mechanisms are not NK− or CTL− (cytotoxic T lymphocyte) specific. Therefore, there is a need for more targeted modulation, which can be accomplished, for example, through modulation of NK and T cell responsiveness mediated through ligands on the surface of NK cells.

NK cells are involved in both the resistance to and control of cancer spread (T. Whiteside and R. Herberman, Current Opinion in Immunology 7:704-710, 1995). Furthermore, the presence and activation of NK cells may be outcome determinative; low or non-existent NK activity is associated with a high frequency of viral disease and cancer (T. Whiteside and R. Herberman, 1995).

As to tumor killing activity, NK cells activated with IL-2 have been shown to have activity against human leukemia cells (L. Silla et al., Journal of Hematotherapy 4:269-279, 1995). Furthermore, NK cells appear to have a role in the treatment of chronic myeloid leukemia (K. Oshimi, 1996).

Host NK cells play an unusual role in bone marrow transplant rejection, as well as solid organ transplant rejection. NK cells cause rejection of parental bone marrow grafts through a phenomenon known as hybrid histocompatibility (L. Lanier, Current Opinion in Immunology 7:626-631, 1995). The effector cells are NK cells and the target antigens are MHC antigens (Id.). In mouse cells, Ly49 molecules present on NK cells bind to MHC class I molecules present on target cells and inhibit NK cell cytotoxic effects (Id.). The hybrid histocompatibility phenomenon can be explained by the heterogeneity of specific Ly49 receptors on NK cells and the lack of a complementary MHC class I molecule on the parental cell (Id.). Since NK cells exert a cytotoxic effect on target cells completely lacking MHC class I molecules, some positive signaling must exist that facilitates the interaction of NK cells with the target cells (Id.).

Similarly, in human NK cells, a receptor family termed the killer cell inhibitory receptors (KIR) has been identified that is MHC class I specific (D. Raulet, Current Opinion in Immunology 8:372-377, 1996). However, the structure of KIRs is entirely different from the Ly49 receptors (D. Raulet, 1996).

Finally, a number of human lymphoproliferative disorders of NK cells are known. These include NK cell-lineage granular lymphocyte proliferative disorder (NK-GLPD), NK cell lymphoma, and acute leukemia of NK cell lineage (K. Oshimi, International Journal of Hematology 63:279-290, 1996). Most patients with aggressive type NK-GLPD die of the disease (K. Oshimi, 1996). NK cell lymphoma is resistant to combination chemotherapy (K. Oshimi, 1996).

With the function of NK cells so important in this variety of physiological responses, there is a need in the art for methods of controlling NK function.

Antibody Against Mouse 2B4 Antigen

The interest in NK cell activity has lead to the generation of monoclonal antibodies that react against mouse NK cells (C. Sentman et al., Hybridoma 8:605-614, 1989). One of these monoclonal antibodies, anti-2B4, reacts specifically with a 66 kDa antigen present on all murine NK cells (C. Sentman et al., 1989).

The 2B4 antigen is also expressed on the surface of a subset of cultured mouse T cells (B. Garni-Wagner et al., J. Immunol. 151:60-70, 1993). Using anti-2B4 to sort cells into 2B4+ and 2B4− populations, it was found that all splenic NK activity can be sorted in the 2B4+ population. The anti-2B4 monoclonal antibody (anti-2B4 mAb) and the anti-2B4 Fab fragments enhance the cytotoxicity of IL-2 stimulated NK cells (B. Garni-Wagner et al., 1993). However, anti-2B4 mAb does not enhance the cell killing of fresh (resting) NK cells (B. Garni-Wagner et al., 1993). In addition, anti-2B4 mAb caused a 10-fold increase in IFN-γ release from activated NK cells (B. Garni-Wagner et al., 1993).

Dendritic epidermal T cells could also be activated by anti-2B4 mAb (G. Schumachers et al., Eur. J. Immunol. 25:1117-1120, 1995; G. Schumachers et al., Journal of Investigative Dermatology 105:592-596, 1995). Therefore, anti-2B4 mAb can be used to modulate the activity and cytotoxicity of murine NK and T cells, and anti-2B4 mAb is commercially available (PharMingen).

The mouse gene encoding the 2B4 antigen was cloned and sequenced (P. Mathew et al., J. Immunol. 151:5328-5337, 1993). The coding sequence of the 2B4 mRNA was deposited in GenBank under the accession number L19057. The cloned gene encodes a protein of 398 amino acids with an 18 amino acid leader and a 24 amino acid transmembrane region (P. Mathew et al., 1993). 2B4 antigen is a member of a family of closely related genes, and a member of the Ig supergene family, that is homologous to murine and rat CD48 and human LFA-3 (P. Mathew et al., 1993). Multiple mRNA are expressed by the mouse 2B4 gene, which are the result of differential splicing of the primary transcript (P. Mathew et al., 1993). Southern blot analysis of human genomic DNA indicated the existence of a human homologue of 2B4; however, RNA blot analysis of RNA from human NK cells suggested that this gene was not expressed in these human cells (P. Mathew et al., 1993).

Recently, 2B4 has been shown to be a counterstructure for mouse CD48 (Brown et al., J. Exp. Med. 188:2083-2090, 1998; and Latchman et al., J. Immunol. 161:5809-5812, 1998).

Antibody Against Human p38 Antigen

Such studies with murine monoclonal antibodies against 2B4 have generated interest in a corresponding human monoclonal antibody, and a monoclonal antibody (designated mAb C1.7) has been identified that recognizes a surface molecule present on all human NK cells and approximately half of CD8+ T cells (N. Valiante and G. Trinichieri, J. Exp. Med. 178:1397-1406, 1993; N. Valiante, U.S. Pat. No. 5,688,690). This antibody is commercially available (Immunotech), and a hybridoma cell line producing the monoclonal antibody was deposited as ATCC HB 11717 (N. Valiante, U.S. Pat. No. 5,688,690). The antibody detects a 38 kD monomeric molecule (p38) in human NK cell lysates by western blot. Stimulation of p38 on NK cells in vitro with the antibody exerts a variety of effects on NK cells. The antibody induces NK cell cytotoxicity against tumor cells including p815, K562, Daudi, THP-1, and virus infected cells; induces the secretion of cytokines, such as IFN-γ and IL-8; modulates NK proliferative responses to stimulation with IL-2 and IL-12; and also induces signaling events, such as Ca++ flux, phosphoinositide turnover, and phospholipase D activation.

The biological effects of stimulation of T cells with the antibody are similar to those for NK cells. The vast majority (˜90%) of cytotoxic T lymphocyte activity against tumor cells was mediated by cells expressing both CD8 and p38 markers. F(ab′)2 fragments of the antibody inhibited non-MHC-restricted cytotoxicity mediated by resting NK cells and rIL-2-cultured T cells. Therefore, the p38 molecule is clearly an important molecule in the activation of NK and T cells.

CD48

CD48 is a membrane glycoprotein found on cells of hematopoietic origin, including T, NK, B cells, monocytes, and granulocytes. It is also known as HuLy-m3, Blast-1, and TCT.1 in humans; sgp-60 and BCM-1 in mice; and OX-45 in rats. CD48 is attached to the cell surface via a glycosylphosphatidylinositol anchor, and can be released from the cell surface by treatment with phospholipase C (Vaughan et al., Immunogenetics 33:113-17, 1991).

cDNA clones for CD48 have been isolated (Vaughan, H. A. et al., Immunogenetics 33: 113-117, 1991). The nucleotide and amino acid sequences of CD48 are known. For example, Genbank accession number M59904 indicates that the amino acid sequence of human CD48 is:

(SEQ ID NO:10)

MWSRGWDSCLALELLLLPLSLLVTSIQGHLVHMTVVSGSNVTLNISESLP
ENYKQLTWFYTFDQKIVEWDSRKSKYFESKFKGRVRLDPQSGALYISKVQ
KEDNSTYIMRVLKKTGNEQEWKIKLQVLDPVPKPVIKIEKIEDMDDNCYL
KLSCVIPGESVNYTWYGDKRPFPKELQNSVLETTLMPHNYSRCYTCQVSN
SVSSKNGTVCLSPPCTLARSFGVEWIASWLVVTVPTILGLLLT

Although the exact biological role of CD48 is not known, stimulation of B cells through CD48 is known to cause enhancement of IL-4, IL-10, and CD40L mediated activation (E. Klyushnenkova et al., Cell Immun. 174:90-98, 1996).

In mice, an anti-CD48 monoclonal antibody inhibited the activation of T cells, resulting in decreased proliferation, IL-2 production, IL-2 receptor expression, and generation of second messengers (Cabrero et al., P.N.A.S. 90:3418-22, 1993). Antibodies to CD48 have also been shown to prolong graft survival in mice and to suppress cell mediated immunity in vivo (Qin et al., J. Exp. Med. 179: 341-6, 1994; Chavin et al., Int. Imm. 6:701-9, 1994).

In other studies, an antibody against human CD48 prevented killing of Epstein-Barr virus (EBV)-transformed B cell by cytotoxic T cells (Del Porto et al., J. Exp. Med. 173:1339-44, 1991). Binding of an anti-CD48 monoclonal antibody to normal peripheral T cells resulted in a Ca²⁺ flux and caused the T cells to become unable to respond to stimulation through the T cell receptor (Thorley-Lawson et al., Biochem. Soc. Trans. 21:976-80, 1993). These cells failed to produce IL-2 and to upregulate IL-2 receptors upon stimulation (Id.). The blockage by anti-CD48 antibodies could be alleviated by addition of IL-2 to the culture (Id.). These experiments indicated that ligation of CD48 on T cells by a receptor molecule might lead to T cell non-responsiveness (Id.).

Interferons upregulate the expression of CD48 on the cell surface (Tissot et al., J. Interferon Cytokine Res. 17:17-26, 1997). In addition, EBV infection upregulates CD48 expression on the cell surface (Fisher et al., Mol. Cell. Biol. 11:1614-23, 1991; Yokoyama et al., J. Immunol. 146:2192-2200, 1991). A soluble form of human CD48 has been detected in plasma, and the level of soluble CD48 is elevated in patients with acute EBV infection, lymphoproliferative disease, and arthritis (Smith et al., J. Clin. Imm. 17:502-9, 1997). DNA polymorphism of CD48 has been seen in healthy controls and rheumatoid arthritis patients, indicating that CD48 is a genetic marker for the manifestation associated with rheumatoid arthritis (Matsui et al., Tissue Antigens 35:203-5, 1990).

Interestingly, injection of an antibody to CD48 resulted in the elimination of a human B cell leukemia in an in vivo mouse model (Sun et al., Clin. Cancer Res. 4:895-900, 1998).

Soluble forms of CD48 have been shown to bind a ligand on epithelial cells, and CD44 has been shown to be involved, but not required for this binding (Ianelli et al., J. Immunol. 159:3910-20, 1997).

In view of the important role that NK and T cells play in vivo in host defenses, tumor cell survival, autoimmune diseases, and transplant rejection, there exists a need in the art for polypeptides and antibodies suitable for use in studies of the modulation of NK, T cell, and B cell activity and in the selection of specific cell types. Further in view of the lack of reagents for the investigation, detection, and modulation of CD48, there exists a need in the art for polypeptides and antibodies suitable for use in studies of CD48.

### SUMMARY OF THE INVENTION

The present invention provides a polypeptide, known as NK Cell Activation Inducing Ligand (NAIL) (previously designated C1.7), which is expressed on cells that include NK cells and certain subpopulations of T cells. The invention encompasses an isolated nucleic acid molecule comprising the DNA sequence of SEQ ID NO:1 and an isolated nucleic acid molecule encoding the amino acid sequence of SEQ ID NO:2, as well as complementary nucleic acids, derivatives, variants, and fragments thereof. The invention also encompasses recombinant vectors that direct the expression of these nucleic acid molecules and host cells transformed or transfected with these vectors.

The invention also encompasses isolated polypeptides and peptides encoded by these nucleic acid molecules, including soluble NAIL polypeptides. The invention further encompasses methods for the production of NAIL polypeptides including culturing a host cell containing a NAIL expression vector, under conditions promoting expression and recovering the polypeptide from the culture medium. Especially, the expression of NAIL polypeptides in bacteria, yeast, plant, and animal cells is encompassed by the invention.

In addition, assays utilizing NAIL polypeptides to screen for NAIL polypeptide counter-structure molecules, potential inhibitors of activity associated with NAIL polypeptide counter-structure molecules, such as CD48, and methods of using NAIL polypeptides, antibodies and NAIL polypeptide counter-structure molecules as therapeutic agents for the treatment of diseases mediated by NAIL polypeptide counter-structure molecules are encompassed by the invention. Further, methods of using NAIL polypeptides in the design of inhibitors thereof are also an aspect of the invention.

Methods of using NAIL and CD48 polypeptides as reagents to detect NAIL and CD48 polypeptides and inhibit the binding of NAIL with CD48 are also an aspect of the invention. The invention also encompasses methods of using NAIL and CD48 polypeptides to stimulate B, NK, and T cells, and to eliminate cancer cells. The invention also includes methods to increase the NAIL-induced release of certain cytokines as well as methods to increase NK cell cytotoxicity.

### BRIEF DESCRIPTION OF THE FIGURES

FIG. 1 presents an a amino acid sequence alignment of HuNAIL and 2B4 (Mu2B4). Potential glycosylation sites within the extracellular domain are marked with asterisks. The predicted transmembrane domain is underlined. The signal sequence cleavage site is indicated by an arrow and was determined by N-terminal sequencing of the purified soluble protein

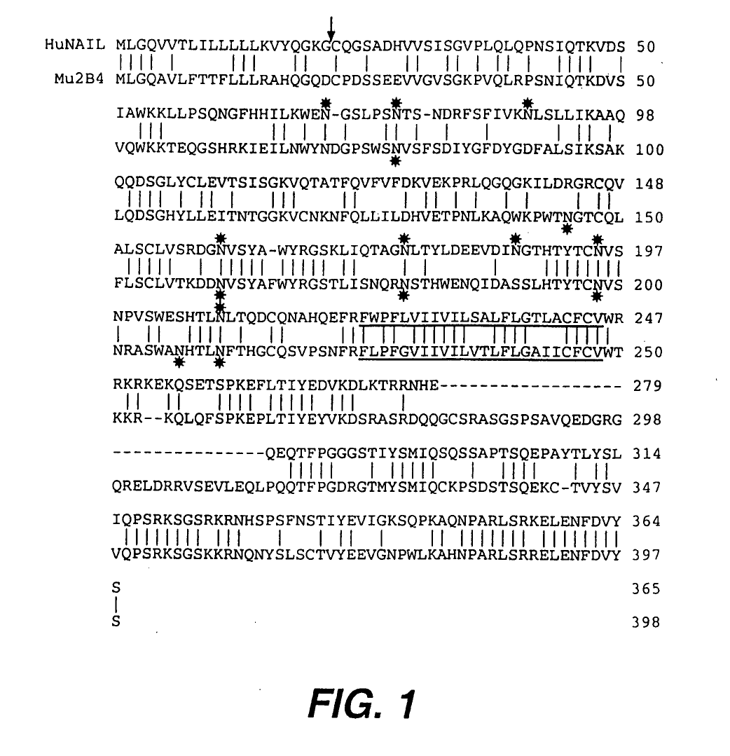

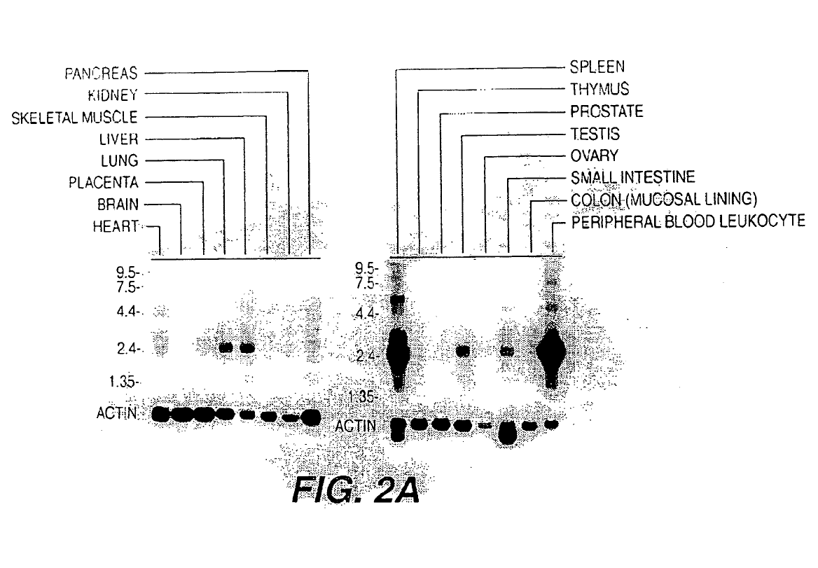

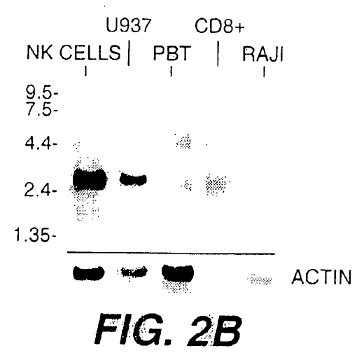

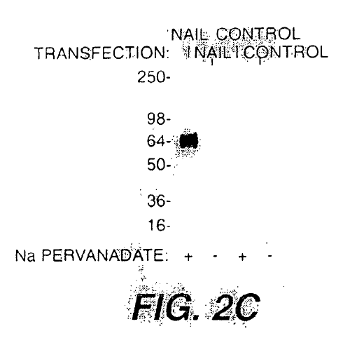

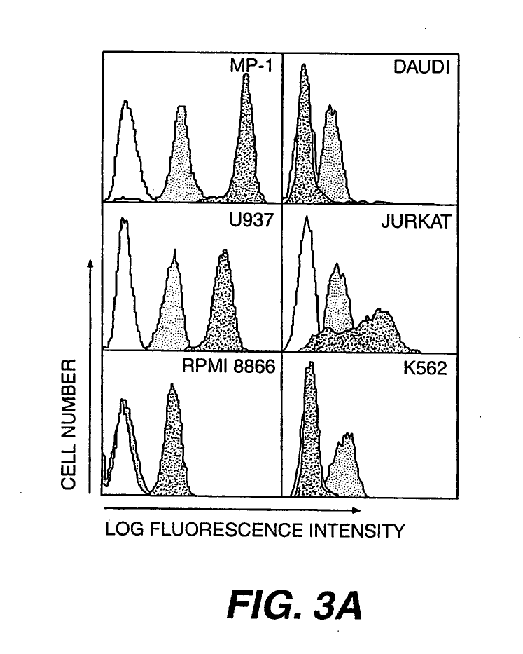

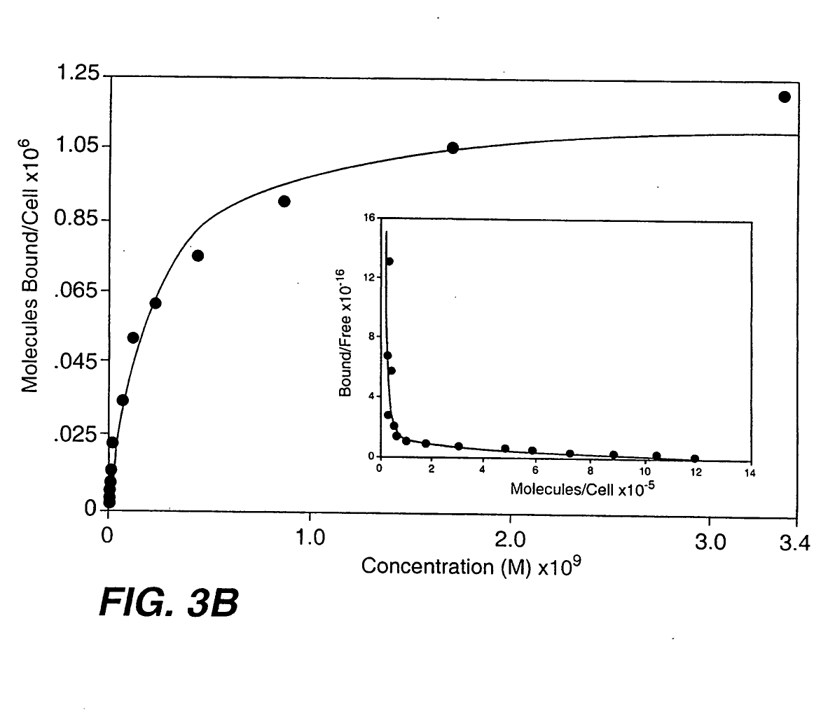

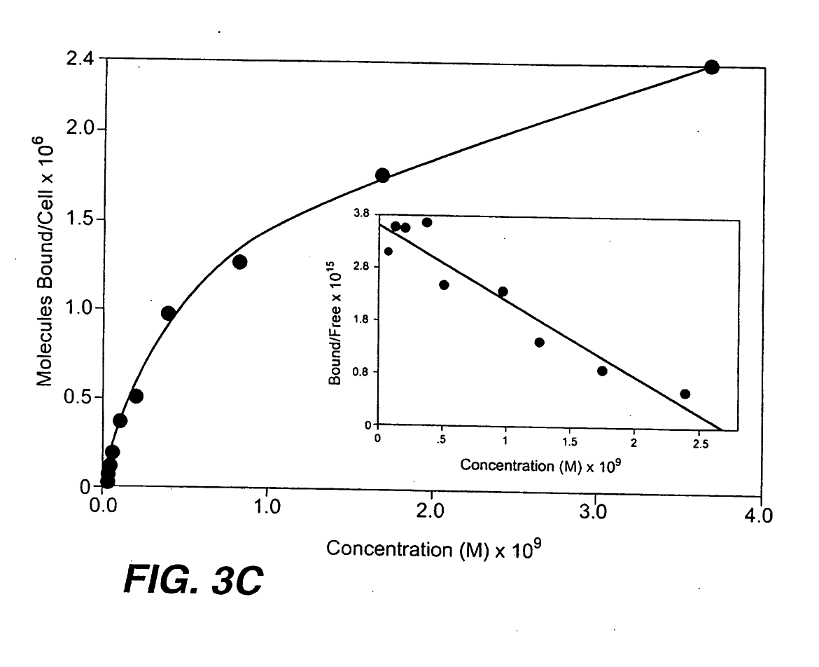

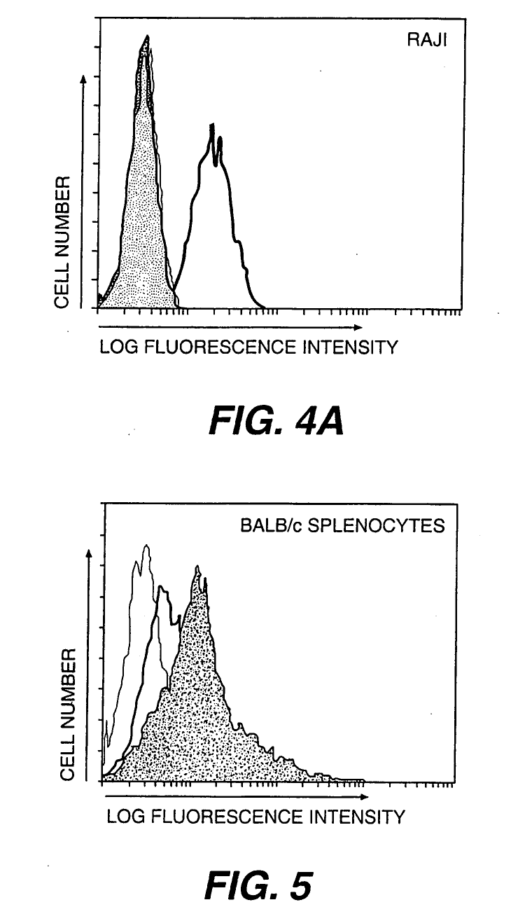

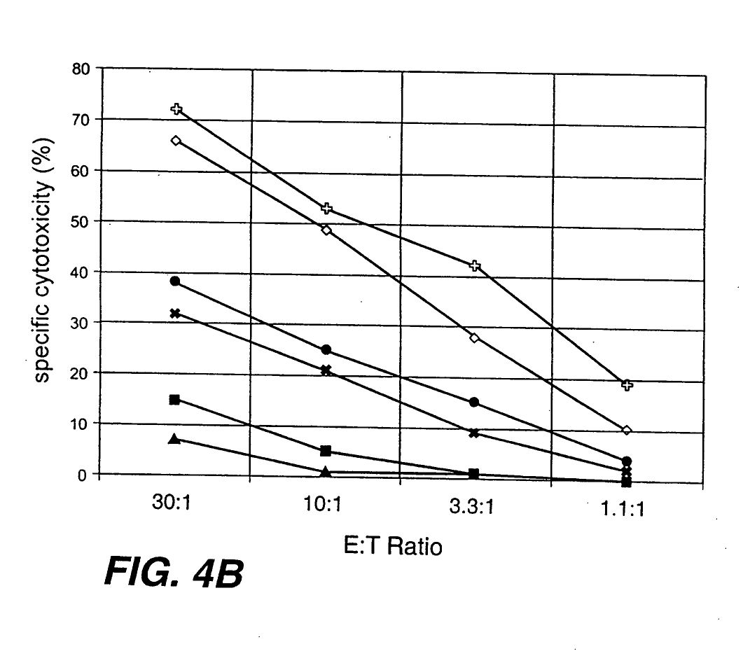

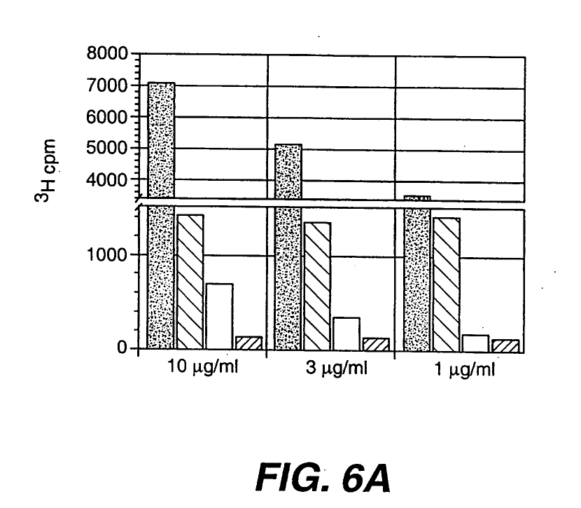

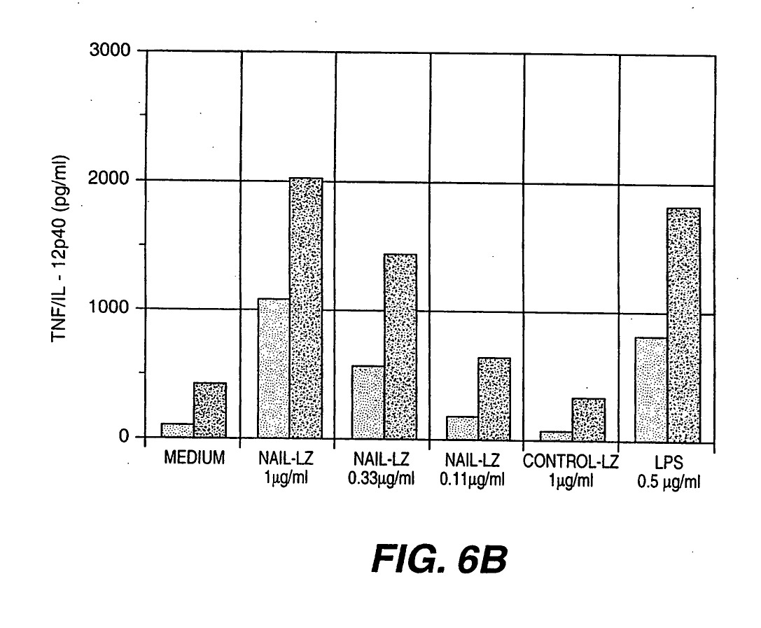

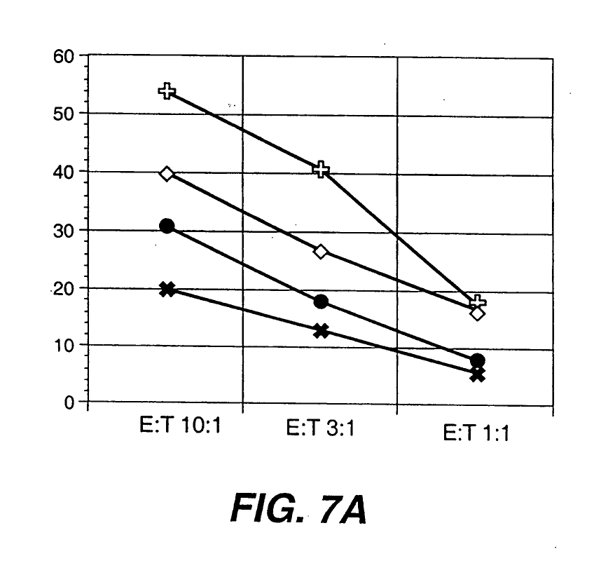

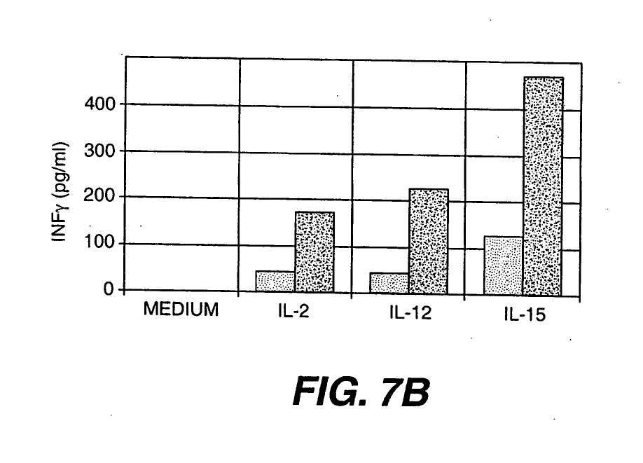

FIG. 2 presents expression of NAIL.
(A) Northern blot analysis of total RNA from indicated tissues. A blot from Clontech containing total RNA was hybridized to a ³²P labeled riboprobe that corresponded to nucleotides 1-890 of NAIL cDNA or ³²P labeled actin cDNA probe, which was labeled by random priming.
(B) Northern blot analysis of total RNA from indicated cells. RNA was electrophoresed on agarose formaldehyde gel, blotted and hybridized to a ³²P labeled riboprobe that corresponded to nucleotides 1-890 of NAIL cDNA or ³²P labeled actin cDNA probe, which was labeled by random priming.
(C) Tyrosine phosphorylation of NAIL. Western blot of anti-phosphotyrosine immunoprecipitates from NAIL-transfected or untransfected CV-1/EBNA cells (control), which had been incubated in the presence or absence of Na prevanadate (+/−). The blot was probed with the anti-C1.7 Mab C1.7.

FIG. 3 presents binding of NAIL to CD48.
(A) Binding of NAIL-Fc to MP-1, Daudi, Jurkat, RPMI-8866, K562, and U937 cell lines. Flow cytometry analysis was performed after incubation of cells in 1 μg/ml of NAIL-Fc (black histogram), negative control p7.5 Fc (white histogram) and IL-17-Fc (gray histogram) fusion proteins, followed by incubation with PE-conjugated goat anti-human Fe Ab.
(B) Various concentrations of NAIL-Fc were incubated with CV-1/EBNA cells transfected with full-length hCD48 cDNA and equilibrium binding determined as described in Example 7. Inset—Scatchard analysis of specific binding.
(C) Various concentrations of CD48-Fc were incubated with CV-1/EBNA cells transfected with full-length NAIL cDNA. Inset—Scatchard analysis of specific binding.

FIG. 4 presents that NAIL and CD48 bind to each other.
(A) FACS analysis of the Raji cell line incubated with NAIL-Fc 5 μg/ml fusion protein in the presence of anti-hCD48 Ab (gray histogram), control Ab (thick line histogram), NAIL Ab (black histogram), control p7.5 Fc fusion protein (thin line histogram).
(B) NK cells were cultured with ⁵¹Cr labeled P815 targets in the presence of 1 μg/ml of anti-NAIL (open diamond) or control anti-CD56 (closed triangle) Ab alone or NAIL Ab in the presence of 10 μg/ml (closed square), 3.3 μg/ml (closed cross) and 1 μg/ml (closed circle) of NAIL-Fc. As a control, NAIL Ab was incubated with 10 μg/ml of a control Fe fusion protein (open cross). ⁵¹Cr release was measured after 4 hours incubation.

FIG. 5 presents that murine CD48 is a counterstructure for 2B4. Mouse splenocytes were incubated in 5 μg/ml of 2B4-Fc in the presence of 10% normal hamster serum (NHS) (black histogram) or anti-mouse CD48 Ab (thick line histogram), or 5 μg/ml of control p7.5 Fc fusion protein (thin line histogram).

FIG. 6 presents the effect of NAIL-CD48 interactions on B and dendritic cells (DC).
(A) Proliferation of peripheral blood B cells. Cells were cultured in the presence of IL-4 (1 ng/ml) (open and hatched bar) or CD40L (300 ng/ml) (black and crosshatched bar) in a 96-well plate precoated with goat anti-human Fc Ab on which NAIL-Fc (black and open bars) or control Fc fusion (crosshatched and hatched bars) proteins at indicated concentrations were immobilized.
(B) DC were stimulated for 48 hours in the presence of indicated concentrations of NAIL-LZ (NAIL-LZ), Control-LZ fusion protein (1 μg/ml) or LPS (0.5 μg/ml). Cell-free supernatants were analyzed for IL-12p40 (gray bars) and TNFα (black bars) content by RIA.

FIG. 7 presents the effect of NAIL-CD48 interaction on NK cells.
(A) NK cells were stimulated with CD48-Fc (open cross and circle) or control Fe (diamond and black cross) fusion proteins (10 μg/ml) immobilized on plates precoated with mouse anti-human Fc polyclonal Ab. Na₂⁵¹CrO₄ labeled Daudi (open cross and diamond) or Raji (circle and black cross) cells were added 1 hour after addition of NK cells at indicated effector:target ratio and ⁵¹Cr release was measured after additional 3 hours of incubation.
(B) IFNγ production by NK cells incubated in the presence of medium, IL-2 (10 ng/ml), IL-12 (1 ng/ml) or IL-15 (50 ng/ml) for 48 hours on plates coated with mouse anti-human Fc on which CD48-Fc (black bar) or control Fc (gray bars) fusion proteins were immobilized. Cell-free supernatants were analyzed for cytokine levels by ELISA.

### DETAILED DESCRIPTION OF THE INVENTION

A cDNA encoding a human polypeptide called NK cell Activation Inducing Ligand (NAIL) (formerly known as C1.7) has been isolated and is disclosed herein. This discovery of the cDNA encoding human NAIL polypeptide enables construction of expression vectors comprising nucleic acid sequences encoding NAIL polypeptides; host cells transfected or transformed with the expression vectors; biologically active human NAIL polypeptide and NAIL as isolated and purified proteins; and antibodies immunoreactive with NAIL polypeptides and peptides.

In making this discovery, cDNA encoding the 38 kd monomeric molecule (p38) on human NK cell lysates (Hup 38) was sequenced, revealing the coding nucleotide sequence of NAIL DNA (SEQ ID NO:1) as well as the nucleotide sequence of NAIL DNA (SEQ ID NO:3). Thus, the invention includes the following NAIL coding sequence:

```
(SEQ ID NO:1)
      1 ATGCTGGGGC AAGTGGTCAC CCTCATACTC CTCCTGCTCC TCAAGGTGTA
    51 TCAGGGCAAA GGATGCCAGG GATCAGCTGA CCATGTGGTT AGCATCTCGG
  101 GAGTGCCTCT TCAGTTACAA CCAAACAGCA TACAGACGAA GGTTGACAGC
  151 ATTGCATGGA AGAAGTTGCT GCCCTCACAA AATGGATTTC ATCACATATT
  201 GAAGTGGGAG AATGGCTCTT TGCCTTCCAA TACTTCCAAT GATAGATTCA
  251 GTTTTATAGT CAAGAACTTG AGTCTTCTCA TCAAGGCAGC TCAGCAGCAG
  301 GACAGTGGCC TCTACTGCCT GGAGGTCACC AGTATATCTG GAAAAGTTCA
  351 GACAGCCACG TTCCAGGTTT TTGTATTTGA TAAAGTTGAG AAACCCCGCC
  401 TACAGGGGCA GGGGAAGATC CTGGACAGAG GGAGATGCCA AGTGGCTCTG
  451 TCTTGCTTGG TCTCCAGGGA TGGCAATGTG TCCTATGCTT GGTACAGAGG
  501 GAGCAAGCTG ATCCAGACAG CAGGGAACCT CACCTACCTG GACGAGGAGG
  551 TTGACATTAA TGGCACTCAC ACATATACCT GCAATGTCAG CAATCCTGTT
  601 AGCTGGGAAA GCCACACCCT GAATCTCACT CAGGACTGTC AGAATGCCCA
  651 TCAGGAATTC AGATTTTGGC CGTTTTTGGT GATCATCGTG ATTCTAAGCG
  701 CACTGTTCCT TGGCACCCTT GCCTGCTTCT GTGTGTGGAG GAGAAAGAGG
  751 AAGGAGAAGC AGTCAGAGAC CAGTCCCAAG GAATTTTTGA CAATTTACGA
  801 AGATGTCAAG GATCTGAAAA CCAGGAGAAA TCACGAGCAG GAGCAGACTT
  851 TTCCTGGAGG GGGGAGCACC ATCTACTCTA TGATCCAGTC CCAGTCTTCT
  901 GCTCCCACGT CACAAGAACC TGCATATACA TTATATTCAT TAATTCAGCC
  951 TTCCAGGAAG TCTGGATCCA GGAAGAGGAA CCACAGCCCT TCCTTCAATA
1001 GCACTATCTA TGAAGTGATT GGAAAGAGTC AACCTAAAGC CCAGAACCCT
1051 GCTCGATTGA GCCGCAAAGA GCTGGAGAAC TTTGATGTTT ATTCC
```

Additional preferred sequences of the invention include nucleotides 57-672 of SEQ ID NO:1.

The invention also includes the full nucleotide sequence of NAIL, as follows:

```
(SEQ ID NO:3)
      1 CGGCCTTGTC AGCTCACAGC AGGCGTTAAC AGCCTCTAAT TGAGGAAACT
    51 GTGGCTGGAC AGGTTGCAAG GCAGTTCTGC TCCCCATCGT CCTCTTGCTG
  101 ACTGGGGACT GCTGAGCCCG TGCACGGCAG AGAGTCTGGT GGGGTGGAGG
  151 GGCTGGCCTG GCCCCTCTGT CCTGTGGAAA TGCTGGGGCA AGTGGTCACC
  201 CTCATACTCC TCCTGCTCCT CAAGGTGTAT CAGGGCAAAG GATGCCAGGG
  251 ATCAGCTGAC CATGTGGTTA GCATCTCGGG AGTGCCTCTT CAGTTACAAC
  301 CAAACAGCAT ACAGACGAAG GTTCACAGCA TTGCATGGAA GAAGTTGCTG
  351 CCCTCACAAA ATGGATTTCA TCACATATTG AAGTGGGAGA ATGGCTCTTT
  401 GCCTTCCAAT ACTTCCAATG ATAGATTCAG TTTTATAGTC AAGAACTTGA
  451 GTCTTCTCAT CAAGGCAGCT CAGCAGCAGG ACAGTGGCCT CTACTGCCTG
  501 GAGGTCACCA GTATATCTGG AAAAGTTCAG ACAGCCACGT TCCAGGTTTT
  551 TGTATTTGAT AAAGTTGAGA AACCCCGCCT ACAGGGGCAG GGGAAGATCC
  601 TGGACAGAGG GAGATGCCAA GTGGCTCTGT CTTGCTTGGT CTCCAGGGAT
  651 GGCAATGTGT CCTATGCTTG GTACAGAGGG AGCAAGCTGA TCCAGACAGC
  701 AGGGAACCTC ACCTACCTGG ACGAGGAGGT TGACATTAAT GGCACTCACA
  751 CATATACCTG CAATGTCAGC AATCCTGTTA GCTGGGAAAG CCACACCCTG
  801 AATCTCACTC AGGACTGTCA GAATGCCCAT CAGGAATTCA GATTTTGGCC
  851 GTTTTTGGTG ATCATCGTGA TTCTAAGCGC ACTGTTCCTT GGCACCCTTG
  901 CCTGCTTCTG TGTGTGGAGG AGAAAGAGGA AGGAGAAGCA GTCAGAGACC
  951 AGTCCCAAGG AATTTTTGAC AATTTACGAA GATGTCAAGG ATCTGAAAAC
1001 CAGGAGAAAT CACGAGCAGG AGCAGACTTT TCCTGGAGGG GGGAGCACCA
1051 TCTACTCTAT GATCCAGTCC CAGTCTTCTG CTCCCACGTC ACAAGAACCT
1101 GCATATACAT TATATTCATT AATTCAGCCT TCCAGGAAGT CTGGATCCAG
1151 GAAGAGGAAC CACAGCCCTT CCTTCAATAG CACTATCTAT GAAGTGATTG
1201 GAAAGAGTCA ACCTAAAGCC CAGAACCCTG CTCGATTGAG CCGCAAAGAG
1251 CTGGAGAACT TTGATGTTTA TTCCTAGTTG CTGCAGCAAT TCTCACCTTT
1301 CTTGCACATC AGCATCTGCT TTGGGAATTG GCACAGTGGA TGACGGCACA
1351 GGAGTCTCTA TAGAACACTT CCTAGTCTGG AGAGGATATG GAAATTTGTT
1401 CTTGTTCTAT ATTTTGTTTT GAAAATGATG TCTAACAACC ATGATAAGAG
1451 CAAGGCTGTT AAATAATATC TTCCAATTTA CAGATCAGAC ATGAATGGGT
1501 GGAGGGGTTA GGTTGTTCAC AAAAGGCCAC ATTCCAAGTA TTTGTAATCT
1551 AGAAAGTGTT ATGTAAGTGA TGTTATTAGC ATCGAGATTC CCTCCACCTG
1601 ATTTTCAAGC TGTCACTTGT TTCCTTTTCT CCCCTCTCTG GGTTGACTGC
1651 ATTTCTAGAC TCTCGCCGGC CCAGGCCCAT CTTCCAAAGC AAGAGGAAGG
1701 AATGATAATG GTGACTCAGG GGAAGAAGAA ACAGCCCTCC TCTGAAAGCC
1751 TGGACTGTCC GGCTGTGAAC TGGCTGGCAG GTTCTGCACG TGGGTGGGGG
1801 CCAGGGCCTG GGCTTTACTC AATTGCAGAG AAAAAACTTT CTCCCTGCAT
1851 CTCATACCTT TACCTCTGGC CAGTTGGCCA CCAGGGGGAG TGGGCTGAAG
1901 GGACAGTAGA TGGTGCAAAG CAAGCCCATC TCTGAGTAGA AAAATCACCC
1951 AGAGCACATG CTGACCTGAT AACTGGGGTG TTGAGACCAG CTTTGTCCAT
2001 GGTATGATGT TTGATTTATG AAGACGCATT GTTAGAAATC CATTTGGCTT
2051 CTTCATAGAA GTGGCTTCCC AGAGGAAGAG GCCTCTCAGA AACCATGTTC
2101 TATTTAAGTT CTGAGTCCTG ATGAGTGTTC CCCAGGATGC ACATTGAAGG
2151 GAGGGCTCAG GCAGCTGAGG GCTGAGAATG AGGCAGTTGG AATCTAGACA
2201 CTATGCTGGG TTCCCTGAGT CGTCAGGCCA GACATTTCAA CAAGGCTGTG
2251 GGGAGCAGGG CTGTGACTCT GGCTGAGCCC AGGAAAGCGA CAAGGGTGAA
2301 CTGGGAGAGG ACTTACTCAG AGACCCCAAC AGGTGATACT GCACAAAGCC
2351 TGGTTCTTCA ATTTTCCTAC CCTGTATCTA ACATAGGAGT TTCATATAAA
2401 ACGGTGATAT CATGCAGATG CAGTCTGAAT TCCTTGCCTG
```

The amino acid sequences of the polypeptides encoded by the nucleotide sequence of the invention include the following:

```
(SEQ ID NO:2)
    1 MLGQVVTLIL LLLLKVYQGK GCQGSADHVV SISGVPLQLQ PNSIQTKVDS
  51 IAWKKLLPSQ NGFHHILKWE NGSLPSNTSN DRFSFIVKNL SLLIKAAQQQ
101 DSGLYCLEVT SISGKVQTAT FQVFVFDKVE KPRLQGQGKI LDRGRCQVAL
151 SCLVSRDGNV SYAWYRGSKL IQTAGNLTYL DEEVDINGTH TYTCNVSNPV
201 SWESHTLNLT QDCQNAHQEF RFWPFLVIIV ILSALFLGTL ACFCVWRRKR
251 KEKQSETSPK EFLTIYEDVK DLKTRRNHEQ EQTFPGGGST IYSMIQSQSS
301 APTSQEPAYT LYSLIQPSRK SGSRKRNESP SFNSTIYEVI GKSQPKAQNP
351 ARLSRKELEN FDVYS
```

The Hup38 clone was used to express recombinant NAIL polypeptide (SEQ ID NO:2) by transfection into CV-1/EBNA cells. By western blot with the commercially available anti-C1.7 monoclonal antibody, the expressed protein was approximately 66 kD in cell lysates. The transfection of CV-1/EBNA cells with Hup38 allowed the cell surface expression of NAIL polypeptides in the transfected cells as determined by slide binding assay, FACS, and Western blot analysis with the C1.7 mAb, described below. The expressed NAIL polypeptide was phosphorylated on tyrosine residues, as determined by immunoprecipitation with anti-phosphotyrosine antibodies.

Expression of the recombinant NAIL polypeptide can also be detected using the C1.7 mAb using other conventional immunological methods as described in Antibodies: A Laboratory Manual, Harlow and Lane (eds.), Cold Spring Harbor Laboratory Press, 1988.

Comparison of the coding nucleotide sequence of NAIL with sequences in GenBank revealed that NAIL was most homologous to the mouse 2B4 sequence (54% amino acid identity and 69% nucleic acid identity). An alignment of human NAIL (top) (SEQ ID NO:2) and mouse 2B4 (bottom) (SEQ ID NO:4) amino acid sequences is as follows:

| | | | |
|---|---|---|---|
| 1 | MLGQVVTLILLLLLKVYQGKGCQGSADHVVSISGVPLQLQPNSIQTKVDS | 50 | (SEQ ID NO:2) |
| | \|\|\|\|.\| :. :\|\|\|:..\|\|.:\|.:\|.:.\|\|::\|\| \|:\|\|.\|..\|\|\|\| \| | | |
| 1 | MLGQAVLFTTFLLLRAHQGQDCPDSSEEVVGVSGKPVQLRPSNIQTKDVS | 50 | (SEQ ID NO:4) |
| | . . . . . | | |
| 51 | IAWKKLLPSQNGFHHILKW..ENGSLPSNTSNDRFSFIVKNLSLLIKAAQ | 98 | |
| | :.\|\|\| .: : .\|\|.\| :..\|:.. . .\| ::\| ::.\| \|\|.\|. | | |
| 51 | VQWKKTEQGSHRKIEILNWYNDGPSWSNVSFSDIYGFDYGDFALSIKSAK | 100 | |
| | . . . . . | | |
| 99 | QQDSGLYCLEVTSISGKVQTATFQVFVFDKVEKPRLQGQGKILDRGRCQV | 148 | |
| | \|\|\|\| \| \|\|:\|..:\|\|\| . .\|\|::::\|.\|\|.\|.\|.:\| \| :..\| \|\|: | | |
| 101 | LQDSGHYLLEITNTGGKVCNKNFQLLILDHVETPNLKAQWKPWTNGTCQL | 150 | |
| | . . . . . | | |
| 149 | ALSCLVSRDGNVSYA.WYRGSKLIQTAGNLTYLDEEVDINGTHTYTCNVS | 197 | |
| | \|\|\|\|\|.:\|:\|\|\|\|\| \|\|\|\|\|.\|\| .. \| \|.:::::\| .: \|\|\|\|\|\|\|\| | | |
| 151 | FLSCLVTKDDNVSYAFWYRGSTLISNQRNSTHWENQIDASSLHTYTCNVS | 200 | |
| | . . . . . | | |
| 198 | NPVSWESHTLNLTQDCQNAHQEFRFWPFLVIIVILSALFLGTLACFCVWR | 247 | |
| | \|..\|\|..\|\|\|\|:\|::\|\|... :\|\|\|:\|\| \|\|\|\|\|\| .\|\|\|\|.: \|\|\|\|\| | | |
| 201 | NRASWANHTLNFTHGCQSVPSNFRFLPFGVIIVILVTLFLGAIICFCVWT | 250 | |
| | . . . . . | | |
| 248 | RKRKEKQSETSPKEFLTIYEDVKDLKTRRNHE.................. | 279 | |
| | :\|\|\|: \| \|\|\|\| \|\|\|\|\| \|\|\| :..\|::: | | |
| 251 | KKRKQLQ..FSPKEPLTIYEYVKDSRASRDQQGCSRASGSPSAVQEDGRG | 298 | |
| | . . . . . | | |
| 280 | ...............QEQTFPGGGSTIYSMIQSQSSAPTSQEPAYTLYSL | 314 | |
| | .:\|\|\|\|\|: :\|:\|\|\|\|\|:..\|..\|\|\|\|. :\|:\|\|: | | |
| 299 | QRELDRRVSEVLEQLPQQTFPGDRGTMYSMIQCKPSDSTSQEK.CTVYSV | 347 | |
| | . . . . . | | |
| 315 | IQPSRKSGSRKRNHSPSFNSTIYEVIGKSQPKAQNPARLSRKELENFDVY | 364 | |
| | :\|\|\|\|\|\|\|\|:\|\|\|:. \|:.:\|:\|\| :\|.. \|\|:\|\|\|\|\|\|\|:\|\|\|\|\|\|\|\| | | |
| 348 | VQPSRKSGSKKRNQNYSLSCTVYEEVGNPWLKAHNPARLSRRELENFDVY | 397 | |
| 365 | S | 365 | |
| | \| | | |
| 398 | S | 398. | |

In this alignment,
a “|” indicates identity of amino acids;
a “.” indicates weak conservation of amino acids, as determined by Bestfit analysis using the UWGCG program; and
a “:” indicates high conservation of amino acids, as determined by Bestfit analysis using the UWGCG program.

An alignment of human NAIL (top) (SEQ ID NO:9) and mouse 2B4 (bottom) (SEQ ID NO:5) nucleotide sequences is as follows:

| | | | |
|---|---|---|---|
| 180 | ATGCTGGGGCAAGTGGTCACCCTCATACTCCTCCTGCTCCTCAAGGTGTA | 229 | (SEQ ID NO:9) |
| | \|\|\| \|\|\|\|\|\|\|\|\| \|\|\| \|\|\| \| \| \|\|\|\|\|\|\|\|\|\|\|\| \|\| \| | | |
| 127 | ATGTTGGGGCAAGCTGTCCTGTTCACAACCTTCCTGCTCCTCAGGGCTCA | 176 | (SEQ ID NO:5) |
| | . . . . . | | |
| 230 | TCAGGGCAAAGGATGCCAGGGATCAGCTGACCATGTGGTTAGCATCTCGG | 279 | |
| | \|\|\|\|\|\|\| \|\|\| \|\|\|\| \| \|\| \|\|\|\| \| \|\|\|\|\|\| \| \|\|\|\| \| | | |
| 177 | TCAGGGCCAAGACTGCCCAGATTCTTCTGAAGAAGTGGTTGGTGTCTCAG | 226 | |
| | . . . . . | | |
| 280 | GAGTGCCTCTTCAGTTACAACCAAACAGCATACAGACGAAGGTTGACAGC | 329 | |
| | \|\| \|\|\|\| \| \|\|\| \| \|\| \|\| \|\|\|\|\|\|\|\|\| \|\| \| \|\| | | |
| 227 | GAAAGCCTGTCCAGCTGAGGCCTTCCAACATACAGACAAAAGATGTTTCT | 276 | |
| | . . . . . | | |
| 330 | ATTGCATGGAAGAAG...TTGCTGCCCTCACAAAATGGATTTCATCACAT | 376 | |
| | \|\| \|\|\|\|\|\|\|\|\|\| \| \| \|\|\|\|\|\| \| \|\| \| \| \|\| | | |
| 277 | GTTCAATGGAAGAAGACAGAACAGGGCTCACACA...GAAAAATTGAGAT | 323 | |
| | . . . . . | | |
| 377 | ATTGAAGTGGGAGAATG......GCTCTTTGCCTTCCAATACTTCCAATG | 420 | |
| | \|\|\|\| \|\|\| \| \|\|\|\| \| \|\| \| \| \|\|\| \| \|\| | | |
| 324 | CCTGAATTGGTATAATGATGGTCCCAGTTGGTCAAATGTATCTTTTAGTG | 373 | |
| | . . . . . | | |
| 421 | ATAGATTCAGTTTTATAGTCAAGAACTTGAGTCTTCTCATCAAGGCAGCT | 470 | |
| | \|\|\| \| \|\|\|\|\| \| \| \|\| \|\|\|\| \|\|\|\|\|\| \|\|\|\|\| | | |
| 374 | ATATCTATGGTTTTGATTATGGGGATTTTGCTCTTAGTATCAAGTCAGCT | 423 | |
| | . . . . . | | |
| 471 | CAGCAGCAGGACAGTGGCCTCTACTGCCTGGAGGTCACCAGTATATCTGG | 520 | |
| | \|\|\| \|\|\| \|\|\|\|\|\|\|\| \| \|\|\|\| \|\|\|\|\|\| \|\|\|\|\|\| \| \| \|\| | | |
| 424 | AAGCTGCAAGACAGTGGTCACTACCTGCTGGAGATCACCAACACAGGCGG | 473 | |
| | . . . . . | | |
| 521 | AAAAGTTCAGACAGCCACGTTCCAGGTTTTTGTATTTGATAAAGTTGAGA | 570 | |
| | \|\|\|\|\|\| \| \| \|\|\|\|\|\| \|\| \|\| \|\| \|\|\|\|\| \| \|\|\|\|\|\|\| | | |
| 474 | AAAAGTGTGCAATAAGAACTTCCAGCTTCTTATACTTGATCATGTTGAGA | 523 | |
| | . . . . . | | |
| 571 | AACCCCGCCTACAGGGGCAGGGGAAGATCCTGGACAGAGGGAGATGCCAA | 620 | |
| | \|\| \|\|\| \|\|\| \|\|\| \|\|\|\|\| \| \| \| \|\|\|\| \|\| \|\|\| | | |
| 524 | CCCCTAACCTGAAGGCCCAGTGGAAGCCCTGGACTAATGGGACTTGTCAA | 573 | |
| | . . . . . | | |
| 621 | GTGGCTCTGTCTTGCTTGGTCTCCAGGGATGGCAATGTGTCCTATGC... | 667 | |
| | \|\| \| \|\|\|\| \|\|\|\|\|\|\|\| \|\|\| \|\|\|\|\| \|\|\|\|\|\|\| \|\|\| \|\| | | |
| 574 | CTGTTTTTGTCCTGCTTGGTGACCAAGGATGACAATGTGAGCTACGCCTT | 623 | |
| | . . . . . | | |
| 668 | TTGGTACAGAGGGAGCAAGCTGATCCAGACAGCAGGGAACCTCACCTACC | 717 | |
| | \|\|\|\|\|\|\|\|\|\|\|\|\|\|\|\|\| \|\|\|\|\|\| \| \| \|\|\|\| \|\|\| \|\| | | |
| 621 | TTGGTACAGAGGGAGCACTCTGATCTCCAATCAAAGGAATAGTACCCACT | 673 | |
| | . . . . . | | |
| 718 | TGGACGAGGAGGTTGACATTAATGGCACTCACACATATACCTGCAATGTC | 767 | |
| | \|\|\| \| \|\| \|\|\|\|\| \| \|\| \|\|\|\|\|\|\|\| \|\|\|\|\|\|\|\| \|\| | | |
| 674 | GGGAGAACCAGATTGACGCCAGCAGCCTGCACACATACACCTGCAACGTT | 723 | |
| | . . . . . | | |
| 768 | AGCAATCCTGTTAGCTGGGAAAGCCACACCCTGAATCTCACTCAGGACTG | 817 | |
| | \|\|\|\|\| \| \|\|\|\|\|\|\| \|\| \|\|\|\|\|\|\|\|\|\|\|\| \|\|\|\| \|\| \| \|\|\| | | |
| 724 | AGCAACAGAGCCAGCTGGGCAAACCACACCCTGAACTTCACCCATGGCTG | 773 | |
| | . . . . . | | |
| 818 | TCAGAATGCCCATCAGGAATTCAGATTTTGGCCGTTTTTGGTGATCATCG | 867 | |
| | \|\|\| \| \|\| \|\| \| \| \| \|\|\|\|\|\|\|\|\| \|\|\| \|\|\| \|\|\|\|\|\|\|\|\|\|\| | | |
| 774 | TCAAAGTGTCCCTTCGAATTTCAGATTTCTGCCCTTTGGGGTGATCATCG | 823 | |
| | . . . . . | | |
| 868 | TGATTCTAAGCGCACTGTTCCTTGGCACCCTTGCCTGCTTCTGTGTGTGG | 917 | |
| | \|\|\|\|\|\|\|\| \|\| \| \|\| \|\| \|\| \|\| \| \|\| \|\|\|\|\|\|\|\|\|\|\|\| | | |
| 824 | TGATTCTAGTTACATTATTTCTCGGGGCCATCATTTGTTTCTGTGTGTGG | 873 | |
| | . . . . . | | |
| 918 | AGGAGAAAGAGGAAGGAGAAGCAGTCAGAGACCAGTCCCAAGGAATTTTT | 967 | |
| | \| \| \|\|\|\|\| \|\|\|\|\|\|\|\| \| \|\| \|\|\| \|\| \|\|\|\|\|\| \|\|\| | | |
| 874 | ACTAAGAAGAG......GAAGCAGTTACAGTTCAGCCCTAAGGAACCTTT | 917 | |
| | . . . . . | | |
| 968 | GACAATTTACGAAGATGTCAAGGATCTGAAAACCAGGAGAAATCA..... | 1012 | |
| | \|\|\|\|\|\| \|\| \|\|\| \|\|\|\|\|\|\|\|\|\| \| \|\|\|\| \|\| \|\|\|\| | | |
| 918 | GACAATATATGAATATGTCAAGGACTCACGAGCCAGCAGGGATCAACAAG | 967 | |
| | . . . . . | | |
| 1013 | ......................................CGAGCAG..... | 1019 | |
| | \|\|\|\|\|\| | | |
| 1018 | GGACAAAGAGAATTGGACAGGCGTGTTTCTGAGGTGCTGGAGCAGTTGCC | 1067 | |
| | . . . . . | | |
| 1020 | .GAGCAGACTTTTCCTGGAGGGGGGAGCACCATCTACTCTATGATCCAGT | 1068 | |
| | \|\|\|\|\|\|\|\|\|\| \|\|\|\|\|\|\| \| \|\|\|\|\|\|\| \|\|\|\|\|\|\|\|\|\|\| \|\|\|\| | | |
| 1068 | ACAGCAGACTTTCCCTGGAGATAGAGGCACCATGTACTCTATGATACAGT | 1117 | |
| | . . . . . | | |
| 1069 | CCCAGTCTTCTGCTCCCACGTCACAAGAACCTGCATATACATTATATTCA | 1118 | |
| | \| \|\| \|\|\|\|\|\| \| \|\|\|\| \|\|\|\|\|\|\|\|\| \|\| \|\|\|\| \|\|\|\|\|\|\|\| | | |
| 1118 | GCAAGCCTTCTGATTCCACATCACAAGAA...AAATGTACAGTATATTCA | 1164 | |
| | . . . . . | | |
| 1119 | TTAATTCAGCCTTCCAGGAAGTCTGGATCCAGGAAGAGGAACCACAGCCC | 1168 | |
| | \|\| \| \|\|\|\|\|\|\|\|\|\|\|\|\|\|\|\|\|\|\|\|\|\|\|\|\| \|\|\|\|\|\|\|\|\|\|\|\| \| \| | | |
| 1165 | GTAGTCCAGCCTTCCAGGAAGTCTGGATCCAAGAAGAGGAACCAGAACTA | 1214 | |
| | . . . . . | | |
| 1169 | TTCCTTCAATAGCACTATCTATGAAGTGATTGGAAAGAGTCAACCTAAAG | 1218 | |
| | \|\|\|\|\|\| \| \| \| \|\| \| \|\| \|\| \| \| \|\|\|\|\|\|\| \| \|\|\|\| | | |
| 1215 | TTCCTTAAGTTGTACCGTGTACGAGGAGGTTGGAAACCCATGGCTCAAAG | 1264 | |
| | . . . . . | | |
| 1219 | CCCAGAACCCTGCTCGATTGAGCCGCAAAGAGCTGGAGAACTTTGATGTT | 1268 | |
| | \| \|\| \|\|\|\|\|\|\|\| \| \|\|\|\|\|\|\|\|\| \|\|\|\|\|\|\|\|\|\|\|\|\|\|\|\|\|\|\|\|\| | | |
| 1265 | CTCACAACCCTGCCAGGCTGAGCCGCAGAGAGCTGGAGAACTTTGATGTC | 1314 | |
| | . . . . . | | |
| 1269 | TATTCCTAG | 1277 | |
| | \|\| \|\|\|\|\|\| | | |
| 1315 | TACTCCTAG 1323 | | |

In light of these alignments, it is evident to the skilled artisan that no contiguous stretch of more than 25 nucleotides or 9 amino acids is conserved between these two sequences.

Nucleic Acid Molecules

In a particular embodiment, the invention relates to certain isolated nucleotide sequences that are free from contaminating endogenous material. A “nucleotide sequence” refers to a polynucleotide molecule in the form of a separate fragment or as a component of a larger nucleic acid construct. The nucleic acid molecule has been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Nucleic acid molecules of the invention include DNA in both single-stranded and double-stranded form, as well as the RNA complement thereof. DNA includes, for example, cDNA, genomic DNA, chemically synthesized DNA, DNA amplified by PCR, and combinations thereof. Genomic DNA may be isolated by conventional techniques, e.g., using the cDNA of SEQ ID NO:1, or a suitable fragment thereof, as a probe.

The DNA molecules of the invention include full length genes as well as polynucleotides and fragments thereof. The full length gene may include the N-terminal signal peptide. Other embodiments include DNA encoding a soluble form, e.g., encoding the extracellular domain of the protein, either with or without the signal peptide.

The nucleic acids of the invention are preferentially derived from human sources, but the invention includes those derived from non-human species, as well.

Preferred Sequences

A particularly preferred nucleotide sequence of the invention is SEQ ID NO:1, as set forth above. A NAIL clone having the nucleotide sequence of SEQ ID NO:1 was isolated as described in Example 1. The sequences of amino acids encoded by the DNA of SEQ ID NO:1 is shown in SEQ ID NO:2. This sequence identifies the NAIL polynucleotide as a member of the Ig superfamily, with closest homology to human CD84 (25% amino acid identity), and CD48 (28% amino acid identity), and murine 2B4 (54% amino acid identity).

The invention further encompasses isolated fragments and oligonucleotides derived from the nucleotide sequence of SEQ ID NO:1, for example by PCR, chemical synthesis, or restriction enzyme digestion. A particularly preferred fragment includes nucleotides 57-672 of SEQ ID NO:1. The invention also encompasses polypeptides encoded by these fragments and oligonucleotides. Different embodiments of the invention include fragments and oligonucleotides that are at least 10-20, 20-30, 30-50, 50-100, 100-300, and 300-1094 nucleotides in size.

Additional Sequences

Due to the known degeneracy of the genetic code, wherein more than one codon can encode the same amino acid, a DNA sequence can vary from that shown in SEQ ID NO:1 and still encode a polypeptide having the amino acid sequence of SEQ ID NO:2. Such variant DNA sequences can result from silent mutations. (e.g., occurring during PCR amplification), or can be the product of deliberate mutagenesis of a native sequence. Exemplary methods of making such alterations are disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

The invention thus provides isolated DNA sequences encoding polypeptides of the invention, selected from: (a) DNA derived from the coding region of a native mammalian NAIL gene; (b) DNA comprising the nucleotide sequence of SEQ ID NO:1; (c) cDNA comprising the nucleotide sequence 1-1095 of SEQ ID NO:1, (d) DNA encoding the polypeptides of SEQ ID NO:2; (e) DNA capable of hybridization to a DNA defined above under conditions of moderate stringency and which encodes polypeptides of the invention; (f) DNA capable of hybridization to a DNA defined above under conditions of high stringency and which encodes polypeptides of the invention; and (g) DNA which is degenerate as a result of the genetic code to a DNA defined above and which encode polypeptides of the invention. Of course, polypeptides encoded by such DNA sequences are encompassed by the invention.

As used herein, conditions of moderate stringency can be readily determined by those having ordinary skill in the art based on, for example, the length of the DNA. The basic conditions are set forth by Sambrook et al. Molecular Cloning: A Laboratory Manual, 2 ed. Vol. 1, pp. 1.101-104, Cold Spring Harbor Laboratory Press, (1989), and include use of a prewashing solution for the nitrocellulose filters 5×SSC, 0.5% SDS, 1.0 mM EDTA (pH 8.0), hybridization conditions of about 50% formamide, 6×SSC at about 42° C. (or other similar hybridization solution, such as Stark's solution, in about 50% formamide at about 42° C.), and washing conditions of about 60° C., 0.5×SSC, 0.1% SDS. Conditions of high stringency can also be readily determined by the skilled artisan based on, for example, the length of the DNA. Generally, such conditions are defined as hybridization conditions as above, and with washing at approximately 68° C., 0.2×SSC, 0.1% SDS. The skilled artisan will recognize that the temperature and wash solution salt concentration can be adjusted as necessary according to factors such as the length of the probe.

The invention also encompasses nucleic acid molecules that hybridize to NAIL DNA (SEQ ID NO:1) under hybridization and wash conditions of 5°, 10°, 15°, 20°, 25°, or 30° below the melting temperature of the DNA duplex of NAIL DNA (SEQ ID NO:1). The invention further encompasses nucleic acid molecules that hybridize to NAIL DNA and are not mouse 2B4 nucleic acid molecules.

In another embodiment, the nucleic acid sequences within the scope of the invention include DNA sequences that vary from SEQ ID NO:1 and encode a polypeptide that specifically binds antibodies against NAIL polypeptide (SEQ ID NO:2).

Also included as an embodiment of the invention is DNA encoding polypeptide fragments and polypeptides comprising inactivated N-glycosylation site(s), inactivated protease processing site(s), or conservative amino acid substitution(s), as described below.

In another embodiment, the nucleic acid molecules of the invention also comprise nucleotide sequences that are at least 80% identical to a native sequence. Also contemplated are embodiments in which a nucleic acid molecule comprises a sequence that is at least 90% identical, at least 95% identical, at least 98% identical, at least 99% identical, or at least 99.9% identical to a native sequence.

The percent identity may be determined by visual inspection and mathematical calculation. Alternatively, the percent identity of two nucleic acid sequences can be determined by comparing sequence information using the GAP computer program, version 6.0 described by Devereux et al. (Nucl. Acids Res. 12:387, 1984) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by one skilled in the art of sequence comparison may also be used.

The invention also provides isolated nucleic acids useful in the production of polypeptides. Such polypeptides may be prepared by any of a number of conventional techniques. A DNA sequence encoding a NAIL polypeptide, or desired fragment thereof, may be subcloned into an expression vector for production of the polypeptide or fragment. The DNA sequence advantageously is fused to a sequence encoding a suitable leader or signal peptide. Alternatively, the desired fragment may be chemically synthesized using known techniques. DNA fragments also may be produced by restriction endonuclease digestion of a full length cloned DNA sequence, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

The well-known polymerase chain reaction (PCR) procedure also may be employed to isolate and amplify a DNA sequence encoding a desired protein fragment. Oligonucleotides that define the desired termini of the DNA fragment are employed as 5′ and 3′ primers. The oligonucleotides may additionally contain recognition sites for restriction endonucleases, to facilitate insertion of the amplified DNA fragment into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et al., eds., Academic Press, Inc. (1990).

Polypeptides and Fragments Thereof

The invention encompasses polypeptides and fragments thereof in various forms, including those that are naturally occurring or produced through various techniques such as procedures involving recombinant DNA technology. Such forms include, but are not limited to, derivatives, variants, and oligomers, as well as fusion proteins or fragments thereof.

As used herein, the term “NAIL polypeptides” refers to a genus of polypeptides that further encompasses proteins having the amino acid sequence 1-365 of SEQ ID NO:2, as well as those proteins having a high degree of similarity (at least 90% identity) with such amino acid sequences and which proteins are biologically active. In addition, NAIL polypeptides refers to the gene products of the nucleotides 1-1095 of SEQ ID NO:1.

The NAIL polypeptides of the invention may be isolated and/or purified or homogeneous. The term “isolated” as used herein, means that the NAIL polypeptides or peptides are substantially separated from the complex mixture of cellular proteins found in its native environment, particularly those proteins of the same molecular weight as native NAIL polypeptides. The term “purified” refers to isolated NAIL polypeptides or peptides, from which other products normally found in the native environment have been substantially removed, particularly those products of the same molecular weight as native NAIL polypeptides.

Thus, “isolated and purified” NAIL polypeptides or peptides are essentially free of other proteins of natural origin, such as a protein that is greater than 95% pure by SDS-PAGE and silver staining. In another embodiment, “isolated and purified” NAIL polypeptides or peptides are recombinant, such as the product of a NAIL expression vector. In another embodiment, “isolated and purified” NAIL polypeptides or peptides are synthesized in vitro. In another embodiment, “isolated and purified” NAIL polypeptides or peptides are essentially free of cellular membrane components. In another embodiment, “isolated and purified” NAIL polypeptides or peptides are essentially free of other proteins, lipids, and carbohydrates found in its native environment. In another embodiment, “isolated and purified” NAIL polypeptides or peptides are homogeneous, essentially free of viruses, bacteria, cellular debris, and cell products.

The expressed full length NAIL polypeptide according to the invention has an observed molecular weight of approximately 66,000 Daltons in CV-1/EBNA cells.

Polypeptides and Fragments Thereof

The polypeptides of the invention include full length proteins encoded by the nucleic acid sequences set forth above. Particularly preferred polypeptides comprise the amino acid sequence of SEQ ID NO:2 with particularly preferred fragments comprising amino acids 22 to 221 of SEQ ID NO:2, which, as described below, make up a soluble version of NAIL.

The polypeptides of the invention may be membrane bound or they may be secreted and thus soluble. Soluble polypeptides are capable of being secreted from the cells in which they are expressed. In general, soluble polypeptides may be identified (and distinguished from non-soluble membrane-bound counterparts) by separating intact cells which express the desired polypeptide from the culture medium, e.g., by centrifugation, and assaying the medium (supernatant) for the presence of the desired polypeptide. The presence of polypeptide in the medium indicates that the polypeptide was secreted from the cells and thus is a soluble form of the protein.

In one embodiment, the soluble polypeptides and fragments thereof comprise all or part of the extracellular domain, but lack the transmembrane region that would cause retention of the polypeptide on a cell membrane as well as lack the cytoplasmic domain. A soluble polypeptide may, however, include the cytoplasmic domain, or a portion thereof, as long as the polypeptide is secreted from the cell in which it is produced.

The NAIL polypeptide comprises a signal peptide (amino acids 1-21 of SEQ ID NO:2), and extracellular domain (amino acids 22-221 of SEQ ID NO:2), a transmembrane domain (amino acids 222-245 of SEQ ID NO:2), and a cytoplasmic domain (amino acids 246-365 of SEQ ID NO:2). The signal sequence cleavage site was determined by N-terminal sequencing of the purified soluble polypeptide. An alternative cleavage site for the signal polypeptide has been predicted after amino acid 18.

Regarding the foregoing discussion of the signal peptide and various domains of the NAIL polypeptide, the skilled artisan will recognize that the above-described boundaries are approximate. The boundaries of the transmembrane region, which may be predicted by using computer programs available for that purpose, may differ from those described above. Thus, soluble NAIL polypeptides, in which the C-terminus of the extracellular domain differs from the residue so identified above, are also encompassed by the invention. As another illustration, cleavage of a signal peptide can occur at sites other than those predicted by computer program. Further, it is recognized that a protein preparation can comprise a mixture of protein molecules having different N-terminal amino acids, due to cleavage of the signal peptide at more than one site.

Soluble polypeptides thus include, but are not limited to, polypeptides comprising amino acids x to 224, wherein x represents any of the amino acids in positions 19 through 22 of SEQ ID NO:2.

Deletion of the leader, cytoplasmic domain, and transmembrane region sequences can be accomplished by conventional molecular techniques, such as those described in Sambrook et al., Molecular Cloning: A Laboratory Manual, 2nd ed., Cold Spring Harbor Laboratory, Cold Spring Harbor, N.Y. (1989), and J. Gatlin et al., BioTechniques 19:599-564, 1995.

In general, the use of soluble forms is advantageous for certain applications. Purification of the polypeptides from recombinant host cells is facilitated, since the soluble polypeptides are secreted from the cells. Further, soluble polypeptides are generally more suitable for intravenous administration.

The invention also provides polypeptides and fragments of the extracellular domain that retain a desired biological activity. Particular embodiments are directed to polypeptide fragments that retain the ability to bind CD48. Such a fragment may be a soluble polypeptide, as described above. In addition, fragments derived from the cytoplasmic domain may find use in studies of signal transduction, and in regulating cellular processes associated with transduction of biological signals. Polypeptide fragments also may be employed as immunogens, in generating antibodies. In another embodiment, the polypeptides and fragments advantageously include regions that are conserved in the NAIL family, as described above.

NAIL polypeptides and peptides of greater than 9 amino acids of SEQ ID NO:2 are an embodiment of the invention, as well as peptides that are at least 10-20, 20-30, 30-50, 50-100, and 100-365 amino acids in size. DNA fragments encoding these polypeptides and peptides are encompassed by the invention.

Synthetic NAIL polypeptides and peptides can be generated by a variety of conventional techniques using the information in SEQ ID NO:2. Such techniques include those described in B. Merrifield, Methods Enzymol. 289:3-13, 1997; H. Ball and P. Mascagni, Int. J. Pept. Protein Res. 48:3147, 1996; F. Molina et al., Pept. Res. 9:151-155, 1996; J. Fox, Mol. Biotechnol. 3:249-258, 1995; and P. Lepage et al., Anal. Biochem. 213: 40-48, 1993.

In another embodiment, NAIL peptides can be prepared by subcloning a DNA sequence encoding a desired NAIL peptide sequence into an expression vector for the production of the desired peptide. The DNA sequence encoding the NAIL peptide is advantageously fused to a sequence encoding a suitable leader or signal peptide. Alternatively, the NAIL DNA fragment may be chemically synthesized using conventional techniques. The NAIL DNA fragment can also be produced by restriction endonuclease digestion of a clone of NAIL DNA, such as Hup38, using known restriction enzymes (New England Biolabs 1997 Catalog, Stratagene 1997 Catalog, Promega 1997 Catalog) and isolated by conventional means, such as by agarose gel electrophoresis. A complete restriction map of NAIL DNA can be obtained using available computer programs. The invention encompasses any and all restriction fragments of NAIL DNA generated with any combination of restriction enzymes, and the encoded peptides. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

In another embodiment, the well known polymerase chain reaction (PCR) procedure can be employed to isolate and amplify a DNA sequence encoding the desired protein fragment. Oligonucleotides that define the desired termini of the DNA fragment are employed as 5′ and 3′ primers. The oligonucleotides can contain recognition sites for restriction endonucleases, to facilitate insertion of the amplified DNA fragments into an expression vector. PCR techniques are described in Saiki et al., Science 239:487 (1988); Recombinant DNA Methology, Wu et al., eds., Academic Press, Inc., San Diego (1989), pp. 189-196; and PCR Protocols: A Guide to Methods and Applications, Innis et al., eds., Academic Press, Inc., (1990). It is understood of course that many techniques could be used to prepare NAIL polypeptide and DNA fragments, and that this embodiment in no way limits the scope of the invention.

Variants

Naturally occurring variants as well as derived variants of the polypeptides and fragments are provided herein. A NAIL polypeptide “variant” as referred to herein means a polypeptide substantially identical to NAIL polypeptide (SEQ ID NO:2), but which has an amino acid sequence different from that of NAIL polypeptide because of one or more deletions, insertions or substitutions. The variant amino acid sequence preferably is at least 80% identical to NAIL polypeptide amino acid sequence (SEQ ID NO:2), most preferably at least 90% identical. Also contemplated are embodiments in which a polypeptide or fragment is at least 90% identical, at least 95% identical, at least 98% identical, at least 99% identical, or at least 99.9% identical to the preferred polypeptide or fragment thereof.

Percent identity may be determined by visual inspection and mathematical calculation. Alternatively, the percent identity of two protein sequences can be determined by comparing sequence information using the GAP computer program, based on the algorithm of Needleman and Wunsch (J. Mol. Bio. 48:443, 1970) and available from the University of Wisconsin Genetics Computer Group (UWGCG). The preferred default parameters for the GAP program include: (1) a scoring matrix, blosum62, as described by Henikoff and Henikoff (Proc. Natl. Acad. Sci. USA 89:10915, 1992); (2) a gap weight of 12; (3) a gap length weight of 4; and (4) no penalty for end gaps. Other programs used by one skilled in the art of sequence comparison may also be used.

The variants of the invention include, for example, those that result from alternate mRNA splicing events or from proteolytic cleavage. Alternate splicing of mRNA may, for example, yield a truncated but biologically active protein, such as a naturally occurring soluble form of the protein. Variations attributable to proteolysis include, for example, differences in the N- or C-termini upon expression in different types of host cells, due to proteolytic removal of one or more terminal amino acids from the protein (generally from 1-5 terminal amino acids). Proteins in which differences in amino acid sequence are attributable to genetic polymorphism (allelic variation among individuals producing the protein) are also contemplated herein.

Additional variants within the scope of the invention include polypeptides that may be modified to create derivatives thereof by forming covalent or aggregative conjugates with other chemical moieties, such as glycosyl groups, lipids, phosphate, acetyl groups and the like. Covalent derivatives may be prepared by linking the chemical moieties to functional groups on amino acid side chains or at the N-terminus or C-terminus of a polypeptide. Conjugates comprising diagnostic (detectable) or therapeutic agents attached thereto are contemplated herein, as discussed in more detail below.

Other derivatives include covalent or aggregative conjugates of the polypeptides with other proteins or polypeptides, such as by synthesis in recombinant culture as N-terminal or C-terminal fusions. Examples of fusion proteins are discussed below in connection with oligomers. Further, fusion proteins can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, Asp-Tyr-Lys-Asp-Asp-Asp-Asp-Lys, which is highly antigenic and provides an epitope reversibly bound by a specific monoclonal antibody, enabling rapid assay and facile purification of expressed recombinant protein. A murine hybridoma designated 4E11 produces a monoclonal antibody that binds the FLAG® peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

In one embodiment, the amino terminal 221 amino acids of NAIL polypeptide have been fused in-frame with Flag and poly-histidine tags, NAIL-Flag-polyHis polypeptide (SEQ ID NO:7). The amino acid sequence of the NAIL-Flag-polyHis polypeptide is as follows:

```
    1 MLGQVVTLIL LLLLKVYQGK GCQGSADHVV SISGVPLQLQ PNSIQTKVDS
  51 IAWKKLLPSQ NGFHHILKWE NGSLPSNTSN DRFSFIVKNL SLLIKAAQQQ
101 DSGLYCLEVT SISGKVQTAT FQVFVFDKVE KPRLQGQGKI LDRGRCQVAL
151 SCLVSRDGNV SYAWYRGSKL IQTAGNLTYL DEEVDINGTH TYTCNVSNPV
201 SWESHTLNLT QDCQNAHQEF RRSGSSDYKD DDDKGSSHHH HHH
```

(SEQ ID NO:7). The Flag tag of the fusion protein is underlined, and the poly-histidine tag of the fusion protein is in bold. Additional amino acid sequences were formed by restriction sites used in the construction of the vector.

Among the variant polypeptides provided herein are variants of native polypeptides that retain the native biological activity or the substantial equivalent thereof. One example is a to variant that binds with essentially the same binding affinity as does the native form. Binding affinity can be measured by conventional procedures, e.g., as described in U.S. Pat. No. 5,512,457 and as set forth below.

Variants include polypeptides that are substantially homologous to the native form, but which have an amino acid sequence different from that of the native form because of one or more deletions, insertions or substitutions. Particular embodiments include, but are not limited to, polypeptides that comprise from one to ten deletions, insertions or substitutions of amino acid residues, when compared to a native sequence.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

The invention further includes polypeptides of the invention with or without associated native-pattern glycosylation. Polypeptides expressed in yeast or mammalian expression systems (e.g., COS-1 or COS-7 cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation may include multiple differentially glycosylated species of the protein. Glycosyl groups can be removed through conventional methods, in particular those utilizing glycopeptidase. In general, glycosylated polypeptides of the invention can be incubated with a molar excess of glycopeptidase (Boehringer Mannheim).

Correspondingly, similar DNA constructs that encode various additions or substitutions of amino acid residues or sequences, or deletions of terminal or internal residues or sequences are encompassed by the invention. For example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in proteins include those described in U.S. Pat. No. 5,071,972 and EP 276,846, hereby incorporated by reference.

In another example of variants, sequences encoding Cys residues that are not essential for biological activity can be altered to cause the Cys residues to be deleted or replaced with other amino acids, preventing formation of incorrect intramolecular disulfide bridges upon folding or renaturation.

Other variants are prepared by modification of adjacent dibasic amino acid residues, to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

Oligomers

Encompassed by the invention are oligomers or fusion proteins that contain NAIL polypeptides. Such oligomers may be in the form of covalently-linked or non-covalently-linked multimers, including dimers, trimers, or higher oligomers. As noted above, preferred polypeptides are soluble and thus these oligomers may comprise soluble polypeptides. In one aspect of the invention, the oligomers maintain the binding ability of the polypeptide components and provide therefor, bivalent, trivalent, etc., binding sites.

One embodiment of the invention is directed to oligomers comprising multiple polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of the polypeptides attached thereto, as described in more detail below.

Immunoglobulin-Based Oligomers

As one alternative, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Proteins”, in Current Protocols in Immunology, Suppl. 4, pages 10.19.1-10.19.11, 1992).

One embodiment of the present invention is directed to a dimer comprising two fusion proteins created by fusing a polypeptide of the invention to an Fc polypeptide derived from an antibody. A gene fusion encoding the polypeptide/Fc fusion protein is inserted into an appropriate expression vector. Polypeptide/Fc fusion proteins are expressed in host cells transformed with the recombinant expression vector, and allowed to assemble much like antibody molecules, whereupon interchain disulfide bonds form between the Fc moieties to yield divalent molecules;

The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred polypeptides comprise an Fc polypeptide derived from a human IgG1 antibody.

One suitable Fc polypeptide, described in PCT application WO 93/10151 (hereby incorporated by reference), is a single chain polypeptide extending from the N-terminal hinge region to the native C-terminus of the Fc region of a human IgG 1 antibody. Another useful Fc polypeptide is the Fc mutein described in U.S. Pat. No. 5,457,035 and in Baum et al., (EMBO J. 13:3992-4001, 1994) incorporated herein by reference. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

The above-described fusion proteins comprising Fc moieties (and oligomers formed therefrom) offer the advantage of facile purification by affinity chromatography over Protein A or Protein G columns.

In other embodiments, the polypeptides of the invention may be substituted for the variable portion of an antibody heavy or light chain. If fusion proteins are made with both heavy and light chains of an antibody, it is possible to form an oligomer with as many as four NAIL extracellular regions.

In one embodiment, a soluble form of the protein can be expressed as an Fc fusion protein. In one embodiment, the amino terminal 221 amino acids of NAIL polypeptide have been fused in-frame with human Fe sequences to generate NAIL-Fc polypeptide (SEQ ID NO:6), using conventional molecular techniques. The amino acid sequence of the NAIL-Fc polypeptide is as follows:

```
(SEQ ID NO:6)
    1 MLGQVVTLIL LLLLKVYQGK GCQGSADHVV SISGVPLQLQ PNSIQTKVDS
  51 IAWKKLLPSQ NGFHHILKWE NGSLPSNTSN DRFSFIVKNL SLLIKAAQQQ
101 DSGLYCLEVT SISGKVQTAT FQVFVFDKVE KPRLQGQGKI LDRGRCQVAL
151 SCLVSRDGNV SYAWYRGSKL IQTAGNLTYL DEEVDINGTH TYTCNVSNPV
201 SWESHTLNLT QDCQNAHQEF RRSCDKTHTC PPCPAPEAEG APSVFLFPPK
251 PKDTLMISRT PEVTCVVVDV SHEDPEVKFN WYVDGVEVHN AKTKPREEQY
301 NSTYRVVSVL TVLHQDWLNG KEYKCKVSNK ALPAPIEKTI SKAKGQPREP
351 QVYTLPPSRE EMTKNQVSLT CLVKGFYPSD IAVEWESNGQ PENNYKTTPP
401 VLDSDGSFFL YSKLTVDKSR WQQGNVFSCS VMHEALHNHY TQKSLSLSPG
451 K.
The Fc portion of the fusion polypeptide is underlined.
```

NAIL-Fc polypeptide was expressed in CV-1/EBNA cells as a soluble polypeptide, using coventional techniques, such as those described in Goodwin et al., Cell 73: 447 (1993). Cells were labeled with ³⁵S-methionine, and cell-free supernatants were resolved by SDS-PAGE. Expression of the soluble fusion protein was detected. It is understood of course that many techniques could be used to isolate soluble NAIL polypeptides, and that this embodiment in no way limits the scope of the invention.

Purification of the soluble fusion protein can be accomplished using the Fc portion of the molecule using conventional techniques, such as those described as in Goodwin et al., Cell 73: 447 (1993). The soluble form of NAIL polypeptide can be used to inhibit the activation of NK cells through the cell-associated molecule by binding to a NAIL counter-structure molecule.

Peptide-Linker Based Oligomers

Alternatively, the oligomer is a fusion protein comprising multiple polypeptides, with or without peptide linkers (spacer peptides). Among the suitable peptide linkers are those described in U.S. Pat. Nos. 4,751,180 and 4,935,233, which are hereby incorporated by reference. A DNA sequence encoding a desired peptide linker may be inserted between, and in the same reading frame as, the DNA sequences of the invention, using any suitable conventional technique. For example, a chemically synthesized oligonucleotide encoding the linker may be ligated between the sequences. In particular embodiments, a fusion protein comprises from two to four soluble NAIL polypeptides, separated by peptide linkers.

Leucine-Zippers

Another method for preparing the oligomers of the invention involves use of a leucine zipper. Leucine zipper domains are peptides that promote oligomerization of the proteins in which they are found. Leucine zippers were originally identified in several DNA-binding proteins (Landschulz et al., Science 240:1759, 1988), and have since been found in a variety of different proteins. Among the known leucine zippers are naturally occurring peptides and derivatives thereof that dimerize or trimerize.

The zipper domain (also referred to herein as an oligomerizing, or oligomer-forming, domain) comprises a repetitive heptad repeat, often with four or five leucine residues interspersed with other amino acids. Examples of zipper domains are those found in the yeast transcription factor GCN4 and a heat-stable DNA-binding protein found in rat liver (C/EBP; Landschulz et al., Science 243:1681, 1989). Two nuclear transforming proteins, fos and jun, also exhibit zipper domains, as does the gene product of the murine proto-oncogene, c-myc (Landschulz et al., Science 240:1759, 1988). The products of the nuclear oncogenes fos and jun comprise zipper domains that preferentially form heterodimer (O'Shea et al., Science 245:646, 1989, Turner and Tjian, Science 243:1689, 1989). The zipper domain is necessary for biological activity (DNA binding) in these proteins.

The fusogenic proteins of several different viruses, including paramyxovirus, coronavirus, measles virus and many retroviruses, also possess zipper domains (Buckland and Wild, Nature 338:547, 1989; Britton, Nature 353:394, 1991; Delwart and Mosialos, AIDS Research and Human Retroviruses 6:703, 1990). The zipper domains in these fusogenic viral proteins are near the transmembrane region of the proteins; it has been suggested that the zipper domains could contribute to the oligomeric structure of the fusogenic proteins. Oligomerization of fusogenic viral proteins is involved in fusion pore formation (Spruce et al, Proc. Natl. Acad. Sci. U.S.A. 88:3523, 1991). Zipper domains have also been recently reported to play a role in oligomerization of heat-shock transcription factors (Rabindran et al., Science 259:230, 1993).

Zipper domains fold as short, parallel coiled coils (O'Shea et al., Science 254:539; 1991). The general architecture of the parallel coiled coil has been well characterized, with a “knobs-into-holes” packing as proposed by Crick in 1953 (Acta Crystallogr. 6:689). The dimer formed by a zipper domain is stabilized by the heptad repeat, designated (abcdefg)ₙ according to the notation of McLachlan and Stewart (J. Mol. Biol. 98:293; 1975), in which residues a and d are generally hydrophobic residues, with d being a leucine, which line up on the same face of a helix. Oppositely-charged residues commonly occur at positions g and e. Thus, in a parallel coiled coil formed from two helical zipper domains, the “knobs” formed by the hydrophobic side chains of the first helix are packed into the “holes” formed between the side chains of the second helix.

The residues at position d (often leucine) contribute large hydrophobic stabilization energies, and are important for oligomer formation (Krystek: et al., Int. J. Peptide Res. 38:229, 1991). Lovejoy et al. (Science 259:1288, 1993) recently reported the synthesis of a triple-stranded α-helical bundle in which the helices run up-up-down. Their studies confirmed that hydrophobic stabilization energy provides the main driving force for the formation of coiled coils from helical monomers. These studies also indicate that electrostatic interactions contribute to the stoichiometry and geometry of coiled coils. Further discussion of the structure of leucine zippers is found in Harbury et al (Science 262:1401, 26 Nov. 1993).

Examples of leucine zipper domains suitable for producing soluble oligomeric proteins are described in PCT application WO 94/10308, as well as the leucine zipper derived from lung surfactant protein D (SPD) described in Hoppe et al. (FEBS Letters 344:191, 1994), hereby incorporated by reference. The use of a modified leucine zipper that allows for stable trimerization of a heterologous protein fused thereto is described in Fanslow et al. (Semin. Immunol. 6:267-278, 1994). Recombinant fusion proteins comprising a soluble polypeptide fused to a leucine zipper peptide are expressed in suitable host cells, and the soluble oligomer that forms is recovered from the culture supernatant.

Certain leucine zipper moieties preferentially form trimers. One example is a leucine zipper derived from lung surfactant protein D (SPD) noted above, as described in Hoppe et al. (FEBS Letters 344:191, 1994) and in U.S. Pat. No. 5,716,805, hereby incorporated by reference in their entirety. This lung SPD-derived leucine zipper peptide comprises the amino acid sequence Pro Asp Val Ala Ser Leu Arg Gin Gln Val Glu Ala Leu Gln Gly Gln Val Gln His Leu Gln Ala Ala Phe Ser Gln Tyr.

Another example of a leucine zipper that promotes trimerization is a peptide comprising the amino acid sequence Arg Met Lys Gln Ile Glu Asp Lys Ile Glu Glu Ile Leu Ser Lys Ile Tyr His Ile Glu Asn Glu Ile Ala Arg Ile Lys Lys Leu Ile Gly Glu Arg, as described in U.S. Pat. No. 5,716,805. In one alternative embodiment, an N-terminal Asp residue is added; in another, the peptide lacks the N-terminal Arg residue.

Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences. Leucine zippers may be derived from naturally occurring leucine zipper peptides, e.g., via conservative substitution(s) in the native amino acid sequence, wherein the peptide's ability to promote oligomerization is retained.

Other peptides derived from naturally occurring trimeric proteins may be employed in preparing trimeric NAIL polypeptides. Alternatively, synthetic peptides that promote oligomerization may be employed. In particular embodiments, leucine residues in a leucine zipper moiety are replaced by isoleucine residues. Such peptides comprising isoleucine may be referred to as isoleucine zippers, but are encompassed by the term “leucine zippers” as employed herein.

In one embodiment, the amino terminal 221 amino acids of NAIL polypeptide has been fused in-frame with leucine zipper (See U.S. Pat. No. 5,716,805) and poly-histidine tags to form NAIL-LZ-polyHis polypeptide (SEQ ID NO:8). The amino acid sequence of the NAIL-LZ-polyHis polypeptide is as follows:

1 MLGQVVTLIL LLLLKVYQGK GCQGSADHVV SISGVPLQLQ PNSIQTKVDS

51 IAWKKLLPSQ NGFHHILKWE NGSLPSNTSN DRFSFIVKNL SLLIKAAQQQ

101 DSGLYCLEVT SISGKVQTAT FQVFVFDKVE KPRLQGQGKI LDRGRCQVAL

151 SCLVSRDGNV SYAWYRGSKL IQTAGNLTYL DEEVDINGTH TYTCNVSNPV

201 SWESHTLNLT QDCQNAHQEF RRSGSSRMKO IEDKIEEILS KIYHIENEIA

251 RIKKLIGERG TSSRGSHHHH HH (SEQ ID NO:8). The leucine zipper tag of the fusion protein is underlined, and the poly-histidine tag of the fusion protein is in bold. Additional amino acid sequences were formed by restriction sites used in the construction of the vector.

Production of Polypeptides and Fragments Thereof

Expression, isolation and purification of the polypeptides and fragments of the invention may be accomplished by any suitable technique, including but not limited to the following:

Expression Systems

The present invention also provides recombinant cloning and expression vectors containing DNA, as well as host cell containing the recombinant vectors. Expression vectors comprising DNA may be used to prepare the polypeptides or fragments of the invention encoded by the DNA. A method for producing polypeptides comprises culturing host cells transformed with a recombinant expression vector encoding the polypeptide, under conditions that promote expression of the polypeptide, then recovering the expressed polypeptides from the culture. The skilled artisan will recognize that the procedure for purifying the expressed polypeptides will vary according to such factors as the type of host cells employed, and whether the polypeptide is membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide upon secretion of polypeptide from the cell.

The skilled artisan will also recognize that the position(s) at which the signal peptide is cleaved may differ from that predicted by computer program, and may vary according to such factors as the type of host cells employed in expressing a recombinant polypeptide. A protein preparation may include a mixture of protein molecules having different N-terminal amino acids, resulting from cleavage of the signal peptide at more than one site. Particular embodiments of mature proteins provided herein include, but are not limited to, proteins having the residue at position 20, 22, 222, 225, or 246 of SEQ ID NO:2 as the N-terminal amino acid and residue at position 221, 224, 245, or 365 of SEQ ID NO:2 as the C-terminal amino acid.

Suitable host cells for expression of polypeptides include prokaryotes, yeast or higher eukaryotic cells. Mammalian or insect cells are generally preferred for use as host cells. Appropriate cloning and expression vectors for use with bacterial, fungal, yeast, and mammalian cellular hosts are described, for example, in Pouwels et al. Cloning Vectors: A Laboratory Manual, Elsevier, New York, (1985). Cell-free translation systems could also be employed to produce polypeptides using RNAs derived from DNA constructs disclosed herein.

Prokaryotic Systems

Prokaryotes include gram-negative or gram-positive organisms. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a polypeptide may include an N-terminal methionine residue to facilitate expression of the recombinant polypeptide in the prokaryotic host cell. The N-terminal Met may be cleaved from the expressed recombinant polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. An appropriate promoter and a DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage λP_{L} promoter and a cI857ts thermolabile repressor sequence. Plasmid vectors available from the American Type Culture Collection which incorporate derivatives of the λP_{L} promoter include plasmid pHUB2 (resident in E. coli strain JMB9, ATCC 37092) and pPLc28 (resident in E. coli RR1, ATCC 53082).

Yeast Systems

Alternatively, the polypeptides may be expressed in yeast host cells, preferably from the Saccharomyces genus (e.g., S. cerevisiae). Other genera of yeast, such as Pichia or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are further described in Hitzeman, EPA-73,657. Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the polypeptide. The α-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 mg/ml adenine and 20 mg/ml uracil.

Yeast host cells transformed by vectors containing an ADH2 promoter sequence may be grown for inducing expression in a “rich” medium. An example of a rich medium is one consisting of 1% yeast extract, 2% peptone, and 1% glucose supplemented with 80 mg/ml adenine and 80 mg/ml uracil. Derepression of the ADH2 promoter occurs when glucose is exhausted from the medium.

Mammalian or Insect Systems

Mammalian or insect host cell culture systems also may be employed to express recombinant polypeptides. Bacculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable host strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534 and PCT Application WO 97/25420) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in WO 91/18982, incorporated by reference herein. In yet another alternative, the vectors can be derived from retroviruses.

Additional useful expression vectors, pFLAG® and pDC311, can also be used. FLAG® technology is centered on the fusion of a low molecular weight (1 kD), hydrophilic, FLAG® marker peptide to the N-terminus of a recombinant protein expressed by pFLAG® expression vectors. pDC311 is another specialized vector used for expressing proteins in CHO cells. pDC311 is characterized by a bicistronic sequence containing the gene of interest and a dihydrofolate reductase (DHFR) gene with an internal ribosome binding site for DHFR translation, an expression augmenting sequence element (EASE), the human CMV promoter, a tripartite leader sequence, and a polyadenylation site.

Regarding signal peptides that may be employed, the native signal peptide may be replaced by a heterologous signal peptide or leader sequence, if desired. The choice of signal peptide or leader may depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal pep tide described in EP 367,566; the type I interleukin-1 receptor signal peptide described in U.S. Pat. No. 4,968,607; and the type II interleukin-1 receptor signal peptide described in EP 460,846.

Purification

The invention also includes methods of isolating and purifying the polypeptides and fragments thereof.

Isolation and Purification

In one preferred embodiment, the purification of recombinant polypeptides or fragments can be accomplished using fusions of polypeptides or fragments of the invention to another polypeptide to aid in the purification of polypeptides or fragments of the invention. Such fusion partners can include the poly-His or other antigenic identification peptides described above as well as the Fc moieties described previously.

With respect to any type of host cell, as is known to the skilled artisan, procedures for purifying a recombinant polypeptide or fragment will vary according to such factors as the type of host cells employed and whether or not the recombinant polypeptide or fragment is secreted into the culture medium.

In general, the recombinant polypeptide or fragment can be isolated from the host cells if not secreted, or from the medium or supernatant if soluble and secreted, followed by one or more concentration, salting-out, ion exchange, hydrophobic interaction, affinity purification or size exclusion chromatography steps. As to specific ways to accomplish these steps, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. In addition, a chromatofocusing step can be employed. Alternatively, a hydrophobic interaction chromatography step can be employed. Suitable matrices can be phenyl or octyl moieties bound to resins. In addition, affinity chromatography with a matrix which selectively binds the recombinant protein can be employed. Examples of such resins employed are lectin columns, dye columns, and metal-chelating columns. Finally, one or more reversed-phase high performance liquid chromatography (RP-HPLC) steps employing hydrophobic RP-HPLC media, (e.g., silica gel or polymer resin having pendant methyl, octyl, octyldecyl or other aliphatic groups) can be employed to further purify the polypeptides. Some or all of the foregoing purification steps, in various combinations, are well known and can be employed to provide an isolated and purified recombinant protein.

It is also possible to utilize an affinity column comprising a polypeptide-binding protein of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention.

In this aspect of the invention, polypeptide-binding proteins, such as the anti-polypeptide antibodies of the invention or other proteins that may interact with the polypeptide of the invention, can be bound to a solid phase support such as a column chromatography matrix or a similar substrate suitable for identifying, separating, or purifying cells that express polypeptides of the invention on their surface. Adherence of polypeptide-binding proteins of the invention to a solid phase contacting surface can be accomplished by any means, for example, magnetic microspheres can be coated with these polypeptide-binding proteins and held in the incubation vessel through a magnetic field. Suspensions of cell mixtures are contacted with the solid phase that has such polypeptide-binding proteins thereon. Cells having polypeptides of the invention on their surface bind to the fixed polypeptide-binding protein and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner.

Alternatively, mixtures of cells suspected of containing polypeptide-expressing cells of the invention first can be incubated with a biotinylated polypeptide-binding protein of the invention. Incubation periods are typically at least one hour in duration to ensure sufficient binding to polypeptides of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The desired degree of purity depends on the intended use of the protein. A relatively high degree of purity is desired when the polypeptide is to be administered in vivo, for example. In such a case, the polypeptides are purified such that no protein bands corresponding to other proteins are detectable upon analysis by SDS-polyacrylamide gel electrophoresis (SDS-PAGE). It will be recognized by one skilled in the pertinent field that multiple bands corresponding to the polypeptide may be visualized by SDS-PAGE, due to differential glycosylation, differential post-translational processing, and the like. Most preferably, the polypeptide of the invention is purified to substantial homogeneity, as indicated by a single protein band upon analysis by SDS-PAGE. The protein band may be visualized by silver staining, Coomassie blue staining, or (if the protein is radiolabeled) by autoradiography.

Use of NAIL Nucleic Acid or Oligonucleotides

In addition to being used to express polypeptides as described above, the nucleic acids of the invention, including DNA, RNA, mRNA, and oligonucleotides thereof can be used:
as probes to identify nucleic acid encoding proteins having NAIL activity; and
as single-stranded sense or antisense oligonucleotides, to inhibit expression of polypeptides encoded by the NAIL gene.

Probes

Among the uses of nucleic acids of the invention is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence.

Because homologs of SEQ ID NO:1 from other mammalian species are contemplated herein, probes based on the DNA sequence of SEQ ID NO:1 may be used to screen cDNA libraries derived from other mammalian species, using conventional cross-species hybridization techniques.

Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified.

Sense-Antisense

Other useful fragments of the nucleic acids include antisense or sense oligonucleotides comprising a single-stranded nucleic acid sequence (either RNA or DNA) capable of binding to target mRNA (sense) or DNA (antisense) sequences. Antisense or sense oligonucleotides, according to the present invention, comprise a fragment of DNA (SEQ ID NO:1). Such a fragment generally comprises at least about 14 nucleotides, preferably from about 14 to about 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block or inhibit protein expression by one of several means, including enhanced degradation of the mRNA by RNAseH, inhibition of splicing, premature termination of transcription or translation, or by other means. The antisense oligonucleotides thus may be used to block expression of proteins. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, lipofection, CaPO₄-mediated DNA transfection, electroporation, or by using gene transfer vectors such as Epstein-Barr virus.

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Use of NAIL Polypeptides and Fragmented Polypeptides

Uses include, but are not limited to, the following:
Assays for activation/inhibition activities
Purification Reagents
Measuring Activity
Delivery Agents
Therapeutic Agents
Research Reagents
Molecular weight and Isoelectric focusing markers
Controls for peptide fragmentation
Identification of unknown proteins
Preparation of Antibodies

Assays for Activation/Inhibition Activities

NAIL polypeptides can be assessed for biological activity based on the ability to induce NK cell activation. Fragments of NAIL polypeptides can be assessed for their ability to mediate this activation, as well as to block native NAIL mediated activation. For example, fragments of NAIL that bind to NAIL mAb can be assessed for their ability to block NAIL mAb mediated activation of cells by conventional titration experiments.

The NAIL polypeptides can be employed in screening for NAIL counter-structure molecules. For example, purified soluble NAIL-Fc fusion protein can be labeled and used to detect cells expressing NAIL counter-structure molecules. Cells expressing NAIL counter-structure molecules on their surface can be screened by methods including slide binding and FACS. If soluble, NAIL counter-structure molecules can be screened by binding to NAIL polypeptide, for example using the NAIL-FC polypeptide bound to an affinity column. In one embodiment, cell supernatants from cells expressing soluble NAIL counter-structure molecules can be passed over a NAIL-Fc polypeptide affinity column. Bound NAIL counter-structure molecules can be detected using conventional techniques. Cells lines expressing soluble NAIL counter-structure molecules can be screened using conventional affinity precipitation techniques to detect NAIL counter-structure molecules in the extracellular supernatant. It is understood of course that many different techniques can be used for the using isolated and purified NAIL polypeptides or peptides to screen for NAIL counter-structure molecules, and that this embodiment in no way limits the scope of the invention.

In another embodiment, the yeast two-hybrid system developed at SUNY (described in U.S. Pat. No. 5,283,173 to Fields et al.; J. Luban and S. Goff., Curr Opin. Biotechnol. 6:59-64, 1995; R. Brachmann and J. Boeke, Curr Opin. Biotechnol. 8:561-568, 1997; R. Brent and R. Finley, Ann. Rev. Genet. 31:663-704, 1997; P. Bartel and S. Fields, Methods Enzymol. 254:241-263, 1995) can be used to screen for a NAIL counter-structure as follows. NAIL, or portions thereof responsible for interaction, can be fused to the Gal4 DNA binding domain and introduced, together with a human cell cDNA library from cells expressing a NAIL counterstructure molecule fused to the Gal 4 transcriptional activation domain, into a strain that depends on Gal4 activity for growth on plates lacking histidine. Interaction of the NAIL polypeptide with a NAIL counter-structure allows growth of the yeast containing both molecules and allows screening for the NAIL counter-structure.

The identification of CD48 as a NAIL counter-structure (Example 2) allows the generation of molecules that can modulate the activation of NK and T cells. Soluble NAIL polypeptide binds to membrane-associated CD48 with high affinity, approximately 10⁻¹⁰M. Conversely, soluble CD48 binds to membrane-associated NAIL (Example 8). Soluble NAIL polypeptide also binds to soluble CD48. In one embodiment, soluble versions of CD48 can be incubated with NK or T cells to enhance or inhibit the induction of NK or T cell activity.

In addition, the identification of CD48 as a NAIL counterstructure allows methods of detecting NAIL and CD48, both soluble and on the surface of cells. For example, by contacting NAIL polypeptide with CD48 and detecting the NAIL/CD48 complex, the level of CD48 can be determined. As indicated in Smith et al., J. Cin. Immunol. 17:502-9 (1997), elevated levels of CD48 may be associated with lymphoid leukemias, arthritis, and EBV infection.

Purified NAIL polypeptides (including proteins, polypeptides, fragments, variants, oligomers, and other forms) may be tested for the ability to bind CD48 in any suitable assay, such as a conventional binding assay. Similarly, CD48 polypeptides (including proteins, polypeptides, fragments, variants, oligomers, and other forms) may be tested for the ability to bind NAIL. To illustrate, the NAIL polypeptide may be labeled with a detectable reagent (e.g., a radionuclide, chromophore, enzyme that catalyzes a colorimetric or fluorometric reaction, and the like). The labeled polypeptide is contacted with cells expressing CD48, such as B cells or dendritic cells. The cells then are washed to remove unbound labeled polypeptide, and the presence of cell-bound label is determined by a suitable technique, chosen according to the nature of the label.

Alternatively, the binding properties of NAIL polypeptides and polypeptide fragments can be determined by analyzing the binding of NAIL polypeptides and polypeptide fragments to cells by FACS analysis as in Example 7. This allows the characterization of the binding of NAIL polypeptides and polypeptide fragments, and the discrimination of relative abilities of NAIL polypeptides and polypeptide fragments to bind to CD48. In vitro binding assays with CD48 can similarly be used to characterize NAIL binding activity.

One example of a binding assay procedure is as follows. A recombinant expression vector containing CD48 cDNA is constructed, e.g., as described in Example 8. DNA and amino acid sequence information for human and mouse CD48 is presented in Staunton et al., EMBO J. 6:3695-3701, 1987, and Cabrero et al., P.N.A.S. 90:3418-3422, 1993. CV1-EBNA-1 cells in 10 cm² dishes are transfected with the recombinant expression vector. CV-1/EBNA-1 cells (ATCC CRL 10478) constitutively express EBV nuclear antigen-1 driven from the CMV immediate-early enhancer/promoter. CV1-EBNA-1 was derived from the African Green Monkey kidney cell line CV-1 (ATCC CCL 70), as described by McMahan et al. (EMBO J. 10:2821, 1991).

The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble NAIL polypeptide/Fc fusion protein made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization.

The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc protein that has bound to the cells. In all assays, non-specific binding of ¹²⁵I-antibody is assayed in the absence of the Fc fusion protein, as well as in the presence of the Fc fusion protein and a 200-fold molar excess of unlabeled mouse anti-human IgG antibody.

Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer.

Another type of suitable binding assay is a competitive binding assay. To illustrate, biological activity of a variant may be determined by assaying for the variant's ability to compete with the native protein for binding to CD48.

Competitive binding assays can be performed by conventional methodology. Reagents that may be employed in competitive binding assays include radiolabeled NAIL and intact cells expressing CD48 (endogenous or recombinant) on the cell surface. For example, a radiolabeled soluble NAIL fragment can be used to compete with a soluble NAIL variant for binding to cell surface CD48. Instead of intact cells, one could substitute a soluble CD48/Fc fusion protein bound to a solid phase through the interaction of Protein A or Protein G (on the solid phase) with the Fc moiety. Chromatography columns that contain Protein A and Protein G include those available from Pharmacia Biotech, Inc., Piscataway, N.J.

Another type of competitive binding assay utilizes radiolabeled soluble CD48, such as a soluble CD48/Fc fusion protein, and intact cells expressing NAIL polypeptide (endogenous or recombinant). Qualitative results can be obtained by competitive autoradiographic plate binding assays, while Scatchard plots (Scatchard, Ann. N.Y. Acad. Sci. 51:660, 1949) may be utilized to generate quantitative results.

NAIL and CD48 polypeptides may also be tested for the ability to exert agonistic effects on cells. For example, NAIL polypeptides, which bind to cell surface CD48, can be assayed for the ability to activate cells through CD48 by contacting a NAIL polypeptide to be tested with cells expressing CD48 and examining the biological consequences of the binding of NAIL to CD48. In one embodiment, stimulation of B cells with NAIL polypeptide is assessed by measuring proliferation of the cells, as in Example 10. This allows the characterization of the activation of cells by NAIL polypeptides and polypeptide fragments through CD48, and the discrimination of relative abilities of NAIL polypeptides and polypeptide fragments to stimulate cells through CD48. In another embodiment, stimulation of dendritic cells with NAIL polypeptide is assessed by measuring the production of cytokines by the cells, as in Example 10. Stimulation of cells with NAIL polypeptide can be assessed by any suitable means, including detection of increased protein production and RNA expression.

Conversely, CD48 polypeptides, which bind to cell surface NAIL, can be assayed for the ability to activate cells through NAIL by contacting a CD48 polypeptide to be tested with cells expressing NAIL and examining the biological consequences of the binding of CD48 to NAIL. In one embodiment, stimulation of NK cells with CD48 polypeptide is assessed by measuring NK cell cytotoxicity, as in Example 11. In another embodiment, stimulation of NK cells with CD48 polypeptide is assessed by measuring cytokine production by the cells, as in Example 11. Stimulation of cells with CD48 can be assessed by any suitable means, including detection of increased protein production and RNA expression.

NAIL and CD48 polypeptides can also be tested for the ability to exert antagonistic effects on cells. That is, NAIL and CD48 polypeptides can be tested for the ability to inhibit the biological effects of NAIL/CD48 binding. For example, a NAIL or CD48 polypeptide can be added to the experimental assay systems described above in a competitive assay. NAIL or CD48 polypeptides, which exhibit antagonistic effects, will compete for binding with the cell surface molecule and inhibit the stimulation of cells that is due to the biological consequences of the binding of NAIL to CD48.

NAIL polypeptides can also be assessed for their ability to inhibit the activation of T cells using the assay systems described in Cabrero et al., P.N.A.S. 90:3418-22, 1993 and Thorley-Lawson et al., Biochem. Soc. Trans. 21:976-80, 1993. NAIL polypeptides can also be assessed for their ability to prolong graft survival and to suppress cell mediated immunity in vivo using the assay systems described in Qin et al., J. Exp. Med. 179: 341-6, 1994, and Chavin et al., Int. Imm. 6:701-9, 1994. NAIL polypeptides can also be assessed for their ability to prevent killing of Epstein-Barr virus (EBV)-transformed B cell by cytotoxic T cells using the assay systems described in Del Porto et al., J. Exp. Med. 173:1339-44, 1991.

Purification Reagents

The polypeptides of the invention find use as a protein purification reagent. For example, the polypeptides may be used to purify CD48 proteins by affinity chromatography. In particular embodiments, a polypeptide (in any form described herein that is capable of binding CD48) is attached to a solid support by conventional procedures. As one example, chromatography columns containing functional groups that will react with functional groups on amino acid side chains of proteins are available (Pharmacia Biotech, Inc., Piscataway, N.J.). In an alternative, a polypeptide/Fc protein (as discussed above) is attached to Protein A- or Protein G-containing chromatography columns through interaction with the Fc moiety.

The polypeptide also finds use in purifying or identifying cells that express CD48 on the cell surface. Polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing CD48 expressing cells are contacted with the solid phase having the polypeptides thereon. Cells expressing CD48 on the cell surface bind to the fixed polypeptides, and unbound cells then are washed away.

Alternatively, the polypeptides can be conjugated to a detectable moiety, then incubated with cells to be tested for CD48 expression. After incubation, unbound labeled matter is removed and the presence or absence of the detectable moiety on the cells is determined.

In a further alternative, mixtures of cells suspected of containing CD48 cells are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (see Berenson, et al. J. Cell. Biochem., 10D:239, 1986). Washing to remove unbound material, and the release of the bound cells, are performed using conventional methods.

NAIL polypeptide-binding proteins, such as the anti-NAIL polypeptide antibodies of the invention or CD48, can be bound to a solid phase such as a column chromatography matrix or a similar substrate suitable for identifying, separating or purifying cells that express NAIL polypeptides on their surface. Adherence of NAIL polypeptide-binding proteins to a solid phase contacting surface can be accomplished by any means, for example, magnetic microspheres can be coated with NAIL polypeptide-binding proteins and held in the incubation vessel through a magnetic field. Suspensions of cell mixtures are contacted with the solid phase that has NAIL polypeptide-binding proteins thereon. Cells having NAIL polypeptides on their surface bind to the fixed NAIL polypeptide-binding protein and unbound cells then are washed away. This affinity-binding method is useful for purifying, screening or separating such NAIL polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner.

Alternatively, mixtures of cells suspected of containing NAIL polypeptide-expressing cells first can be incubated with a biotinylated CD48. Incubation periods are typically at least one hour in duration to ensure sufficient binding to NAIL polypeptides. The resulting mixture then is passed through a column packed with avidin-coated beads, whereby the high affinity of biotin for avidin provides the binding of the NAIL polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

In another embodiment, CD48 polypeptides may be attached to a solid support material and used to purify NAIL polypeptides by affinity chromatography.

Measuring Activity

Polypeptides also find use in measuring the biological activity of CD48 protein in terms of their binding affinity. The polypeptides thus may be employed by those conducting “quality assurance” studies, e.g., to monitor shelf life and stability of protein under different conditions. For example, the polypeptides may be employed in a binding affinity study to measure the biological activity of a CD48 protein that has been stored at different temperatures, or produced in different cell types. The proteins also may be used to determine whether biological activity is retained after modification of a CD48 protein (e.g., chemical modification, truncation, mutation, etc.). The binding affinity of the modified CD48 protein is compared to that of an unmodified CD48 protein to detect any adverse impact of the modifications on biological activity of CD48. The biological activity of a CD48 protein thus can be ascertained before it is used in a research study, for example.

Delivery Agents

The polypeptides also find use as carriers for delivering agents attached thereto to cells bearing CD48 or NAIL. Cells expressing CD48 include B cells, T cells, and dendritic cells. Cells expressing NAIL include NK and T cells. The polypeptides thus can be used to deliver diagnostic or therapeutic agents to such cells (or to other cell types found to express CD48, or NAIL, on the cell surface) in in vitro or in vivo procedures.

Detectable (diagnostic) and therapeutic agents that may be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating proteins, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu.

Such agents may be attached to the polypeptide by any suitable conventional procedure. The polypeptide comprises functional groups on amino acid side chains that can be reacted with functional groups on a desired agent to form covalent bonds, for example. Alternatively, the protein or agent may be derivatized to generate or attach a desired reactive functional group. The derivatization may involve attachment of one of the bifunctional coupling reagents available for attaching various molecules to proteins (Pierce Chemical Company, Rockford, Ill.). A number of techniques for radiolabeling proteins are known. Radionuclide metals may be attached to polypeptides by using a suitable bifunctional chelating agent, for example.

Conjugates comprising polypeptides and a suitable diagnostic or therapeutic agent (preferably covalently linked) are thus prepared. The conjugates are administered or otherwise employed in an amount appropriate for the particular application.

Therapeutic Agents

Polypeptides of the invention may be used in developing treatments for any disorder mediated (directly or indirectly) by defective or insufficient amounts of the polypeptides. These polypeptides may be administered to a mammal afflicted with such a disorder.

Isolated and purified NAIL and CD48 polypeptides and peptides can also be useful themselves as a therapeutic agent to inhibit NK and T cell signaling, as well as to inhibit NAIL-mediated or CD48-mediated disorders.

The polypeptides may also be employed in inhibiting a biological activity of NAIL and CD48, in in vitro or in vivo procedures. For example, a purified polypeptide may be used to inhibit binding of endogenous NAIL to endogenous CD48. In one embodiment, NAIL polypeptide may be administered to a mammal to treat a CD48-mediated disorder. Such CD48-mediated disorders include conditions caused (directly or indirectly) or exacerbated by CD48. In another embodiment, soluble NAIL polypeptide can be administered to a patient in an effective amount to compete with the binding of endogenous NAIL with CD48, thereby interfering with normal signaling through endogenous NAIL. Alternatively, CD48 polypeptide may be administered in vivo to a patient afflicted with a NAIL-mediated disorder.

Chelation

The binding of soluble NAIL to soluble CD48 allows methods of chelating CD48 and inhibiting the binding of CD48 with NAIL polypeptide on the cell surface. “Chelation” as referred to herein with respect to CD48 means binding to soluble CD48 that neutralizes the ability of the bound soluble CD48 to bind to membrane bound CD48 counterstructures. The chelation of CD48 and the inhibition of natural CD48/NAIL binding should permit the modulation of the immunological effects of this binding, for example, NK and T cell activation.

In one embodiment, a patient's blood or plasma is contacted with NAIL polypeptide ex vivo. The NAIL polypeptide may be bound to a suitable chromatography matrix by conventional procedures. The patient's blood or plasma flows through a chromatography column containing NAIL polypeptide bound to the matrix, before being returned to the patient. The immobilized NAIL polypeptide binds soluble CD48, thus removing soluble CD48 protein from the patient's blood.

Alternatively, NAIL polypeptides may be administered in vivo to a patient afflicted with a CD48-mediated disorder, for example, to chelate soluble CD48. In one embodiment, a soluble form of NAIL is administered to the patient, and chelates soluble CD48, preventing the activation of NK cells by the soluble CD48 molecules. In another embodiment, a soluble form of NAIL is administered to a patient with rheumatoid arthritis in an amount sufficient to chelate elevated levels of soluble CD48 in the patient. NAIL polypeptides and polypeptide fragments capable of binding to soluble CD48 can be assessed, for example, by competition with the binding of labeled soluble human CD48 to cells expressing NAIL on the cell surface as described in Example 8. Other competitive binding assays could similarly be used to assess binding activity of NAIL polypeptides and polypeptide fragments.

In one embodiment, NAIL polypeptides and polypeptide fragments, which bind to soluble and cell surface CD48, can be used. In another embodiment, NAIL polypeptides and polypeptide fragments, which bind to soluble CD48, but do not bind to membrane bound CD48, are used. In another embodiment, NAIL polypeptides and polypeptide fragments, which bind to soluble and membrane associated CD48, but do not stimulate cells through CD48, are used. NAIL polypeptides and polypeptide fragments can be assessed for binding and stimulatory activity using the assays described in the Examples.

The corollary of each of these embodiments, in which CD48 polypeptides are used in place of NAIL polypeptides is also part of this invention. The assessment of CD48 polypeptides and polypeptide can be undertaken by competition with the binding and activation of cells by soluble human CD48 as described in Example 11.

B Cells

In another embodiment, soluble NAIL polypeptides can be used to stimulate B cells through CD48, for example, using the conditions in Klyshnenkova et al., 1996, and in Example 10, particularly in the presence of soluble human CD40L, IL-4, or IL-10. B cells can be incubated with soluble NAIL polypeptides to enhance B cell proliferation and the production of cytokines and IgM. NAIL polypeptides and polypeptide fragments capable of stimulating B cells through CD48 can be assessed, for example, by in vitro assays as described in Example 10. Other assays could similarly be used to assess stimulatory activity of NAIL polypeptides and polypeptide fragments. In one embodiment, a soluble form of NAIL is administered to the patient in an amount sufficient to stimulate B cells. Consequently, soluble NAIL polypeptides can serve as an adjuvant in combination with vaccines.

The binding of soluble NAIL to soluble CD48 allows methods of inhibiting the binding of membrane-bound CD48 with NAIL polypeptide. The inhibition of natural CD48/NAIL binding should permit the modulation of the immunological effects of this binding, for example, B cell activation.

In another embodiment, a soluble form of CD48 is administered to the patient, and binds to NAIL, preventing the activation of B cells by the endogenous NAIL molecules.

Dendritic Cells

In another embodiment, soluble NAIL polypeptides can be used to stimulate dendritic cells through CD48 to produce IL-12p40 and TNF-α. NAIL polypeptides and polypeptide fragments capable of stimulating dendritic cells through CD48 can be assessed, for example, by in vitro assays as described in Example 10. Other assays could similarly be used to assess stimulatory activity of NAIL polypeptides and polypeptide fragments. In one embodiment, a soluble form of NAIL is administered to the patient in an amount sufficient to stimulate dendritic cells to produce IL-12p40 and TNF-α.

The binding of soluble NAIL to soluble CD48 allows methods of inhibiting the binding of membrane-bound CD48 with NAIL polypeptide. The inhibition of natural CD48/NAIL binding should permit the modulation of the immunological effects of this binding, for example, dendritic cell activation. In another embodiment, a soluble form of CD48 is administered to the patient, and binds to NAIL, preventing the activation of dendritic cells by the endogenous NAIL molecules.

NK and T Cells

In another embodiment, soluble human CD48 can be used to stimulate NK and cytotoxic T cells through NAIL polypeptide, for example, using the conditions in Valiante et al., 1993, and Example 11, which can result in increased cytotoxicity against tumor cells and virus infected cells. NK and cytotoxic T cells can be incubated with soluble CD48 polypeptides to stimulate NK and cytotoxic T cells through NAIL polypeptide and increase production of cytokines, such as IFN-γ and IL-8, which play an essential role in antiviral responses, activation of antigen presenting cells, generation of cytotoxic T lymphocytes, and other inflammatory responses. CD48 polypeptides and polypeptide fragments capable of stimulating NK and cytotoxic T cells through NAIL polypeptide can be assessed, for example, by in vitro assays as described in Example 11. Other assays could similarly be used to assess stimulatory activity of CD48 polypeptides and polypeptide fragments.

In another embodiment, NAIL polypeptides can also be used to inhibit the activation of NK and T cells, as described in Cabrero et al., P.N.A.S. 90:3418-22, 1993, and Thorley-Lawson et al., Biochem. Soc. Trans. 21:976-80, 1993. In one embodiment, a soluble form of NAIL polypeptide is administered to the patient in an amount sufficient to inhibit the activation of T cells.

Immune Responses

NAIL polypeptides can also be used to prolong graft survival and to suppress cell mediated immunity in vivo, as described in Qin et al., J. Exp. Med. 179: 341-6, 1994, and Chavin et al., Int. Imm. 6:701-9, 1994. In one embodiment, a soluble form of NAIL polypeptide is administered to the patient in an amount sufficient to suppress cell mediated immunity in vivo. In another embodiment, a soluble form of NAIL polypeptide is administered to the patient in an amount sufficient to prolong graft survival. In a further embodiment, a soluble form of NAIL polypeptide is administered to the patient together with anti-CD2 antibodies or a CD2 counterstructure in an amount sufficient to prolong graft survival.

Cytotoxicity

In another embodiment, soluble NAIL polypeptides can be used to inhibit the proliferation of cancer cells and EBV infected cells through binding to CD48, for example, as in Sun et al., Clin. Cancer Res. 4:895-900, 1998. Soluble NAIL polypeptides can be incubated with cancer cells expressing CD48 to modulate this effect.

In another embodiment, a soluble NAIL-Fc fusion protein is used, which exhibits a high affinity for Fc receptors.

In this regard, NAIL can bind to CD48 on lymphoma and leukemia cells. Therefore, NAIL polypeptide can be attached to a toxin or made radioactive to kill the tumor cells expressing CD48. The methodology can be similar to the successful use of an anti-CD72 immunotoxin to treat therapy-refractory B-lineage acute lymphoblastic leukemia in SCID mice (Meyers et al., Leuk and Lymph. 18:119-122).

NAIL and CD48 may also be employed in conjunction with other agents useful in treating a particular disorder.

Compositions

Compositions of the present invention may contain a polypeptide in any form described herein, such as native proteins, variants, derivatives, oligomers, and biologically active fragments. In particular embodiments, the composition comprises a soluble polypeptide or an oligomer comprising soluble NAIL or CD48 polypeptides.

Compositions comprising an effective amount of a polypeptide of the present invention, in combination with other components such as a physiologically acceptable diluent, carrier, or excipient, are provided herein. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa.

In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance, and are thus chosen according to the intended application.

The compositions of the invention can be administered in any suitable manner, e.g., topically, parenterally, or by inhalation. The term “parenteral” includes injection, e.g., by subcutaneous, intravenous, or intramuscular routes, also including localized administration, e.g., at a site of disease or injury. Sustained release from implants is also contemplated. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration is performed according to art-accepted practices.

Compositions comprising nucleic acids in physiologically acceptable formulations are also contemplated. DNA may be formulated for injection, for example.

Research Reagents

Another use of the polypeptide of the present invention is as a research tool for studying the biological effects that result from inhibiting NAIL/CD48 interactions on different cell types. Polypeptides also may be employed in in vitro assays for detecting CD48 or NAIL or the interactions thereof.

Another embodiment of the invention relates to uses of NAIL polypeptides to study cell signal transduction. NAIL, like other NK cell receptors, could play a central role in immune responses which includes cellular signal transduction, activation of B and NK cells, and production of cytokines. As such, alterations in the expression and/or activation of NAIL can have profound effects on a plethora of cellular processes. Expression of cloned NAIL, functionally inactive mutants of NAIL, or the extracellular or intracellular domain can be used to identify the role a particular protein plays in mediating specific signaling events.

Cellular signaling often involves a molecular activation cascade, during which a receptor propagates a ligand-receptor mediated signal by specifically activating intracellular kinases which phosphorylate target substrates. These substrates can themselves be kinases which become activated following phosphorylation. Alternatively, they can be adaptor molecules that facilitate down stream signaling through protein-protein interaction following phosphorylation. Regardless of the nature of the substrate molecule(s), expressed functionally active versions of NAIL, for example the extracellular or intracellular domain of NAIL, can be used in assays such as the yeast 2-hybrid assay to identify what substrate(s) were recognized and activated by the NAIL binding partner(s). As such, these novel NAIL polypeptides can be used as reagents to identify novel molecules involved in signal transduction pathways.

NAIL DNA, NAIL polypeptides, and antibodies against NAIL polypeptides can be used as reagents in a variety of research protocols. A sample of such research protocols are given in Sambrook et al. Molecular Cloning: A Laboratory Manual, 2 ed. Vol. 1-3, Cold Spring Harbor Laboratory Press, (1989). For example, these reagents can serve as markers for cell specific or tissue specific expression of RNA or proteins. Similarly, these reagents can be used to investigate constitutive and transient expression of NAIL RNA or polypeptides. NAIL DNA can be used to determine the chromosomal location of NAIL DNA and to map genes in relation to this chromosomal location. NAIL DNA can also be used to examine genetic heterogeneity and heredity through the use of techniques such as genetic fingerprinting, as well as to identify risks associated with genetic disorders. NAIL DNA can be further used to identify additional genes related to NAIL DNA and to establish evolutionary trees based on the comparison of sequences. NAIL DNA and polypeptides can be used to select for those genes or proteins that are homologous to NAIL DNA or polypeptides, through positive screening procedures such as Southern blotting and immunoblotting and through negative screening procedures such as subtraction.

NAIL polypeptides can also be used as a reagent to identify (a) any protein that NAIL polypeptide regulates, and (b) other proteins with which it might interact. NAIL polypeptides could be used by coupling recombinant protein to an affinity matrix, or by using them as a bait in the 2-hybrid system. NAIL polypeptides and peptides can be used as reagents in the study of the NK and T cell signaling pathways to block NK and T cell signaling. Antibodies directed against NAIL polypeptides can be used as reagents in the study of the NK and T cell signaling pathways to inhibit or activate NK and T cell signaling.

The purified NAIL polypeptides according to the invention will facilitate the discovery of inhibitors of NAIL polypeptides. The use of a purified NAIL polypeptide in the screening of potential inhibitors thereof is important and can eliminate or reduce the possibility of interfering reactions with contaminants.

In addition, NAIL polypeptides can be used for structure-based design of NAIL polypeptide-inhibitors. Such structure-based design is also known as “rational drug design.” The NAIL polypeptides can be three-dimensionally analyzed by, for example, X-ray crystallography, nuclear magnetic resonance or homology modeling, all of which are well-known methods. The use of NAIL polypeptide structural information in molecular modeling software systems to assist in inhibitor design and inhibitor-NAIL polypeptide interaction is also encompassed by the invention. Such computer-assisted modeling and drug design can utilize information such as chemical conformational analysis, electrostatic potential of the molecules, protein folding, etc. For example, most of the design of class-specific inhibitors of metalloproteases has focused on attempts to chelate or bind the catalytic zinc atom. Synthetic inhibitors are usually designed to contain a negatively-charged moiety to which is attached a series of other groups designed to fit the specificity pockets of the particular protease. A particular method of the invention comprises analyzing the three dimensional structure of NAIL polypeptides for likely binding sites of substrates, synthesizing a new molecule that incorporates a predictive reactive site, and assaying the new molecule as described above.

Identification of Unknown Proteins

As set forth above, a polypeptide or peptide fingerprint can be entered into or compared to a database of known proteins to assist in the identification of the unknown protein using mass spectrometry (W. J. Henzel et al., Proc. Natl. Acad. Sci. USA 90:5011-5015, 1993; D. Fenyo et al., Electrophoresis 19:998-1005, 1998). A variety of computer software programs to facilitate these comparisons are accessible via the Internet, such as Protein Prospector (Internet site: prospector.uscf.edu), MultiIdent (Internet site: www.expasy.ch/sprot/multiident.html), PeptideSearch (Internet site:www.mann.embl-heiedelberg.de...deSearch/FR^{—}PeptideSearchForm.html), and ProFound (Internet site:www.chait-sgi.rockefeller.edu/cgi-bin/prot-id-frag.html). These programs allow the user to specify the cleavage agent and the molecular weights of the fragmented peptides within a designated tolerance. The programs compare observed molecular weights to predicted peptide molecular weights derived from sequence databases to assist in determining the identity of the unknown protein.

In addition, a polypeptide or peptide digest can be sequenced using tandem mass spectrometry (MS/MS) and the resulting sequence searched against databases (J. K. Eng, et al., J. Am. Soc. Mass Spec. 5:976-989 (1994); M. Mann and M. Wilm, Anal. Chem. 66:4390-4399 (1994); J. A. Taylor and R. S. Johnson, Rapid Comm. Mass Spec. 1: 1067-1075 (1997)). Searching programs that can be used in this process exist on the Internet, such as Lutefisk 97 (Internet site: www.lsbc.com:70/Lutefisk97.html), and the Protein Prospector, Peptide Search and ProFound programs described above.

Therefore, adding the sequence of a gene and its predicted protein sequence and peptide fragments to a sequence database can aid in the identification of unknown proteins using mass spectrometry.

Antibodies

Immunogenic NAIL polypeptides and peptides are encompassed by the invention. The immunogenicity of NAIL peptides and polypeptides can be determined by conventional techniques, such as those described in Antibodies: A Laboratory Manual, Harlow and Lane (eds.), Cold Spring Harbor Laboratory Press, 1988. Within an aspect of the invention, NAIL polypeptides, and peptides based on the amino acid sequence of NAIL, can be utilized to prepare antibodies that specifically bind to NAIL polypeptides.

Antibodies that are immunoreactive with the polypeptides of the invention are provided herein. In this aspect of the invention, the polypeptides based on the amino acid sequence of NAIL can be utilized to prepare antibodies that specifically bind to NAIL. Such antibodies specifically bind to the polypeptides via the antigen-binding sites of the antibody (as opposed to non-specific binding). Thus, the polypeptides, fragments, variants, fusion proteins, etc., as set forth above may be employed as immunogens in producing antibodies immunoreactive therewith. More specifically, the polypeptides, fragment, variants, fusion proteins, etc. contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes may be identified by any of the methods known in the art.

Thus, one aspect of the present invention relates to the antigenic epitopes of the polypeptides of the invention. Such epitopes are useful for raising antibodies, in particular monoclonal antibodies, as described in detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies may be prepared by conventional techniques as described below.

The term “antibodies” is meant to include polyclonal antibodies, monoclonal antibodies, fragments thereof, particularly antigen binding fragments such as F(ab′)2 and Fab fragments, as well as any recombinantly produced binding partners. Antibodies are defined to be specifically binding if they bind with a Kₐ of greater than or equal to about 10⁷ M⁻¹. Affinities of binding partners or antibodies can be readily determined using conventional techniques, for example those described by Scatchard et al., Ann. N.Y Acad Sci., 51:660 (1949).

Polyclonal antibodies can be readily generated from a variety of sources, for example, horses, cows, goats, sheep, dogs, chickens, rabbits, mice, or rats, using procedures that are well known in the art. In general, purified NAIL or a peptide based on the amino acid sequence of NAIL polypeptide that is appropriately conjugated is administered to the host animal typically through parenteral injection. The immunogenicity of NAIL polypeptide can be enhanced through the use of an adjuvant, for example, Freund's complete or incomplete adjuvant. Following booster immunizations, small samples of serum are collected and tested for reactivity to NAIL polypeptide. Examples of various assays useful for such determination include those described in Antibodies: A Laboratory Manual, Harlow and Lane (eds.), Cold Spring Harbor Laboratory Press, 1988; as well as procedures, such as countercurrent immuno-electrophoresis (CIEP), radioimmunoassay, radio-immunoprecipitation, enzyme-linked immunosorbent assays (ELISA), dot blot assays, and sandwich assays. See U.S. Pat. Nos. 4,376,110 and 4,486,530.

Monoclonal antibodies can be readily prepared using well known procedures. See, for example, the procedures described in U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.), 1980. Briefly, the host animals, such as mice, are injected intraperitoneally at least once and preferably at least twice at about 3 week intervals with isolated and purified NAIL, optionally in the presence of adjuvant. Mouse sera are then assayed by conventional dot blot technique or antibody capture (ABC) to determine which animal is best to fuse. Approximately two to three weeks later, the mice are given an intravenous boost of NAIL or conjugated NAIL peptide. Mice are later sacrificed and spleen cells fused with commercially available myeloma cells, such as Ag8.653 (ATCC), following established protocols. Briefly, the myeloma cells are washed several times in media and fused to mouse spleen cells at a ratio of about three spleen cells to one myeloma cell. The fusing agent can be any suitable agent used in the art, for example, polyethylene glycol (PEG). Fusion is plated out into plates containing media that allows for the selective growth of the fused cells. The fused cells can then be allowed to grow for approximately eight days. Supernatants from resultant hybridomas are collected and added to a plate that is first coated with goat anti-mouse Ig. Following washes, a label, such as ¹²⁵I-NAIL, is added to each well followed by incubation. Positive wells can be subsequently detected by autoradiography. Positive clones can be grown in bulk culture and supernatants are subsequently purified over a Protein A column (Pharmacia).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3:1-9 (1990), which is incorporated herein by reference. Similarly, binding partners can be constructed using recombinant DNA techniques to incorporate the variable regions of a gene that encodes a specific binding antibody. Such a technique is described in Larrick et al., Biotechnology, 7:394 (1989).

The monoclonal antibodies of the present invention include chimeric antibodies, e.g., humanized versions of murine monoclonal antibodies. Such humanized antibodies may be prepared by known techniques, and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806 and related patents claiming priority therefrom, all of which are incorporated by reference herein.

Uses Thereof

Once isolated and purified, the antibodies against NAIL polypeptides and other NAIL binding proteins can be used to detect the presence of NAIL polypeptides in a sample using established assay protocols. Further, the antibodies of the invention can be used therapeutically to bind to NAIL polypeptides and inhibit its activity in vivo.

Antibodies directed against NAIL polypeptides and other NAIL binding proteins can be used to modulate the activity of NK and T cells using techniques such as those described in N. Valiante and G. Trinichieri, J. Exp. Med. 178:1397-1406, 1993; N. Valiante, U.S. Pat. No. 5,688,690. One class of these antibodies can activate NK and T cell activity similar to the “C1.7 mAb” (Immunotech). In contrast, another class of these antibodies can inhibit a biological activity mediated through NAIL polypeptide. In one embodiment, antibodies inhibit NK and T cell activation through NAIL polypeptide, for example, by interfering with the interaction of NAIL polypeptide and its counter-structure.

Antibodies directed against NAIL polypeptides and other NAIL binding proteins can also be used to purify specific subtypes of cells expressing NAIL polypeptide by conventional methods including FACS and panning techniques, such as those described in Antibodies: A Laboratory Manual, Harlow and Lane (eds.), Cold Spring Harbor Laboratory Press, 1988 and Merwe et al., Eur J. Immunol. 23:1373-1377, 1993.

Antibodies directed against NAIL polypeptides and other NAIL binding proteins can also be used to determine the levels of populations of NAIL-positive cells by conventional methods including flow cytometry.

Such antibodies and other NAIL binding proteins can also be useful in the diagnosis of pathological states the result in overexpression or underexpression of NAIL polypeptides, such as in cancers and autoimmune diseases.

It is also understood that whether an antibody interacts with the same epitope as “C1.7 mAb” (N. Valiante, U.S. Pat. No. 5,688,690) can readily be determined by conventional techniques, such as epitope mapping and antibody competition studies. For example, an antibody that binds to an epitope other than that bound by “C1.7 mAb” can bind to a NAIL polypeptide fragment, which does not bind to “C1.7 mAb”. Polyclonal and monoclonal antibodies that do not bind to the same epitope as “C1.7 mAb” (N. Valiante, U.S. Pat. No. 5,688,690) are encompassed by this invention.

Those antibodies that additionally can block NAIL/CD48 binding of may be used to is inhibit a biological activity that results from such binding. Such blocking antibodies may be identified using any suitable assay procedure, such as by testing antibodies for the ability to inhibit binding of NAIL to cells expressing CD48, or to inhibit binding of CD48 to certain cells expressing NAIL. Antibodies may be assayed for the ability to inhibit CD48-mediated stimulation of B cells or dendritic cells, for example. Alternatively, blocking antibodies may be identified in assays for the ability to inhibit a biological effect that results from binding of NAIL to target cells.

Such an antibody may be employed in an in vitro procedure, or administered in vivo to inhibit a biological activity mediated by the entity that generated the antibody. Disorders caused or exacerbated (directly or indirectly) by the interaction of CD48 with cell surface NAIL receptor thus may be treated. A therapeutic method involves in vivo administration of a blocking antibody to a mammal in an amount effective in inhibiting a NAIL or CD48-mediated biological activity. Monoclonal antibodies are generally preferred for use in such therapeutic methods. In one embodiment, an antigen-binding antibody fragment is employed.

Antibodies may be screened for agonistic (i.e., ligand-mimicking) properties. Such antibodies, upon binding to cell surface NAIL or CD48, induce biological effects (e.g., transduction of biological signals) similar to the biological effects induced when CD48 binds to NAIL. Agonistic antibodies may be used to induce NAIL-mediated stimulation of NK and T cells, or to induce CD48-mediated stimulation of B cells and dendritic cells.

Compositions comprising an antibody that is directed against NAIL or CD48, and a physiologically acceptable diluent, excipient, or carrier, are provided herein. Suitable components of such compositions are as described above for compositions containing NAIL or CD48 proteins.

Also provided herein are conjugates comprising a detectable (e.g., diagnostic) or therapeutic agent, attached to the antibody. Examples of such agents are presented above. The conjugates find use in in vitro or in vivo procedures.

The specification is most thoroughly understood in light of the teachings of the references cited within the specification which are hereby incorporated by reference. The embodiments within the specification and the examples provide an illustration of embodiments of the invention and should not be construed to limit the scope of the invention. The skilled artisan readily recognizes that many other embodiments are encompassed by the invention.

### EXAMPLE 1

### Cloning of NAIL DNA

A clone (Hup38) containing the NAIL cDNA was selected from a cDNA expression library by a panning protocol described in van der Merwe et al., Eur J. Immunol. 23:1373-1377, 1993. The expression library was generated using pooled mRNAs extracted from unstimulated human NK cells and human NK cells stimulated with known activators (IL-2, IL-12, IL-15, IFN-γ, and anti-CD16 antibody) for 4 or 14 hours. Double-stranded cDNA was generated from polyA-selected RNA using reverse transcriptase with both random primers and oligo dT. The double-stranded cDNA was cloned into the mammalian expression vector pDC409 using the adaptor procedure as described in R. Goodwin et al., Cell 73:447-456, 1993.

After library production, pools of 10,000 clones were spread on Luria Broth agar plates containing ampicillin. The colonies from each plate were collected by scraping the plates, and plasmid DNA was prepared from one-third of the harvested bacteria using a mini-prep procedure. The remaining bacteria were frozen in glycerol. 10 cm plates of CV-1/EBNA cells were transfected with 500 nanograms of DNA from these pools using the DEAE-dextran procedure.

Three days post-transfection, the cells were dissociated from the plate using cell dissociation buffer (Sigma). These cells were pelleted and resuspended in binding media with 5% non-fat dried milk. After a 30 minute incubation on ice, 0.15 mg of magnetic beads linked to C1.7 mAb via sheep anti-mouse antibody was added to the cells. This antibody is commercially available (Immunotech), and a hybridoma cell line producing the monoclonal antibody was deposited as ATCC HB 11717 (N. Valiante, U.S. Pat. No. 5,688,690). The magnetic beads, precoated with sheep anti-mouse antibody, were obtained from Dynal (cat. # 112.01). The mixture was incubated at 4° C. for 1 hour, with rotation. The beads were separated from the non-bound cells using a magnet, and subjected to six washes with binding media. After the last wash, the beads were placed into a 24 well plate and examined by microscopy. Two pools appeared positive as determined by the visualization of 10-20 cells in each pool coated with magnetic beads.

Positive pools were confirmed by slide binding using C1.7 mAb, followed by a goat anti-mouse antibody labeled with ¹²⁵I, using the technique described in D. Gearing et al., EMBO J. 8:3667-3676, 1989. The isolation of the specific clone, Hup38, was achieved by slide binding, and the ability of the clone to bind C1.7 mAb was reconfirmed by slide binding.

### EXAMPLE 2

Identification of CD48 as a Counterstructure of NAIL Polypeptide

A clone expressing a protein, which was capable of binding to NAIL polypeptide was isolated from a cDNA expression library by a panning protocol described in van der Merwe et al., Eur. J. Immunol. 23:1373-77 (1993). The expression library was prepared from mRNA isolated from the human monocytic cell line U937, and was constructed in the expression vector pDC302 using the adaptor procedure described in R. Goodwin et al., Cell 73:447-56 (1993).

Pools of 2000 clones were spread on Luria Broth agar plates containing ampicillin. The colonies from each plate were collected by scraping the plates, and plasmid DNA was prepared from one-third of the harvested bacteria using a mini-prep procedure. The remaining bacteria were frozen in glycerol. 10 cm plates of CV-1/EBNA cells were transfected with 500 nanograms of DNA from these pools using the DEAE-dextran procedure.

Three days post-transfection, the cells were dissociated from the plate using cell dissociation buffer (Sigma). These cells were pelleted and resuspended in binding media with 5% non-fat dried milk. After a 30 minute incubation on ice, 0.15 mg of magnetic beads linked to NAIL-Fc polypeptide via a goat antibody specific for the Fc portion of human IgG1 was added to the cells. Streptavidin-coated magnetic beads (Dynal; cat. # 112.05) were first bound tobiotinylated goat anti-human IgG-Fc antibody (Jackson Immunoresearch; #109-065-098). After a wash, purified NAIL-Fc polypeptide was then bound to the beads via the bound antibody, followed by a wash.

The mixture was incubated at 4° C. for 1 hour, with rotation. The beads were separated from the non-bound cells using a magnet, and subjected to six washes with binding media. After the last wash, the beads were placed into a 24 well plate and examined by microscopy. Six pools out of 25 tested appeared positive as determined by the visualization of 8-80 cells in each pool coated with magnetic beads.

Positive pools were confirmed by slide binding using NAIL-Fc polypeptide, followed by a goat anti-human IgG1 antibody labeled with ¹²⁵I, using the technique described in D. Gearing et al., EMBO J. 8:3667-3676 (1989). One of the positive pools was then broken down into smaller pools of clones and screened by the slide binding method, ultimately yielding a pure clone which expressed the protein specifically bound by NAIL-Fc polypeptide. Sequencing of the cDNA in this clone revealed that it was identical to CD48.

### EXAMPLE 3

### Generation of Soluble cDNA Constructs and Fusion Proteins

Full length hCD48 was cloned into the pDC409 mammalian expression vector by PCR using the isolated cDNA as a template. A soluble form of CD48 was constructed by fusing the extracellular domain (amino acids 1-216) to the Fc portion of a human mutein IgG1 sequence as previously described (Smith et al., Cell 73:1349-1360, 1993). The CD48-Fc encoding sequences were then inserted into the mammalian expression vector pDC412, a derivative of pDC409 (Wiley et al., Immunity 3:673-682, 1996). Alternative forms of CD48 were constructed in which the C-terminal Fc was replaced with either Flag-poly His (Hopp et al, Biotechnology 6:1204-1210, 1988) or a leucine zipper-poly His tag (Morris et al., J. Biol. Chem., in press, 1999). Soluble NAIL polypeptide was constructed by amplifying the extracellular domain (amino acids 1-221) by PCR using the isolated cDNA as a template. The PCR product was then subcloned into pDC412 in frame with a C-terminal Flag-poly His tag or a leucine zipper-poly His tag (Morris et al., J. Biol. Chem., in press, 1999).

Purification of Fc fusion proteins was performed as described (Goodwin et al., Eur. J. Immunol. 23:2631-2641, 1993). Briefly, columns were packed with POROS 20A from Perspective Biosystems (Farmingham, Mass.), prewashed with PBS pH7.4 PFM (buffer A) followed by 50 mM Na citrate pH 3.0 PFM (buffer B) and equilibrated with buffer A.

Culture supernatants from cells transfected with Fc fusion protein cDNA were loaded on equilibrated column washed with buffer A after which bound protein was eluted with buffer B and collected in 1 ml fractions which were immediately neutralized using 1.5 M HEPES pH 11.0. Collected fractions were run on SDS-PAGE gel, peak fractions pooled, dialyzed at 4° C. overnight against buffer A using 7000 MWCO dialysis tubing and filtered through 0.22 μm Centrex filter (Schleicher & Schuell, Dasel, Germany). Protein concentration was determined by AAA assay and tested for possible endotoxin contamination using LAL assay (Sigma) and was below 5 pg/μg of protein.

Purification of LZ-PH tagged proteins was performed on Ailtech column packed with Ni-NTA Superflow resin according to manufacturer's suggestion (Quiagen, Valencia, Calif.). Protein concentration and endotoxin levels were performed in the same way as for Fc fusion proteins.

### EXAMPLE 4

### Tissue Distribution of NAIL RNA

Northern blot analysis was performed on various tissue RNA using a ³²P-labeled RNA probe encompassing nucleotides 1-890 of the NAIL cDNA (FIG. 2A). Northern blot analysis of RNA samples was performed by using Clontech (Palo Alto, Calif.) multiple tissue Northern blots I and II, or by resolving 10 μg of each total RNA on 1.1% agarose formaldehyde gel and blotting onto Hybond-N as recommended by the manufacturer (Amersham Corp., Arlington Heights, Ill.). The 5′ end of the NAIL cDNA clone (nucleotides 1-890) was subcloned into pBlueScript (Stratagene, La Jolla, Calif.) which, after being linearized with SalI, was used as a template to generate an antisense RNA probe using T3 RNA polymerase. A random primed ³²P-labeled DNA probe was used to monitor the actin level in each RNA sample (Stratagene).

The highest expression of mRNA for NAIL was found in RNA extracted from spleen and peripheral blood lymphocytes (PBL) followed by lung, liver, testis and small intestine. Smaller, yet detectable levels of NAIL mRNA were seen in heart, placenta, pancreas, colon, kidney and ovary. No NAIL message was detected in brain, skeletal muscle, thymus or prostate. Several bands of approximately 2.6, 3.2, 4.8 and 7.5 kb were detected. This heterogeneity was most pronounced in the spleen and PBL were NAIL message was expressed at much higher levels than in any other tissues.

Northern blot analysis of various cells of hemopoietic origin revealed the highest level of NAIL mRNA expression in NK cells and the monocytic cell line U937 followed by CD8⁺ cells and total PBT (FIG. 2B). In this case, two prominent transcripts of 2.6 and 4.4 kb were detected. Detection of NAIL mRNA correlated well with the surface expression of NAIL protein as assessed by FACS analysis using NAIL. The relatively low levels of NAIL mRNA in PBL could be explained by a low percentage (approx. 10-15%) of cells expressing NAIL protein on the cell surface. Purified CD8⁺T cells expressed higher levels of NAIL mRNA than total PBL, which correlates well with the observation that approximately 50% of these cells are positive for NAIL surface expression. NK cells and the U937 cell line, 100% of which have high levels of NAIL protein on the surface, expressed the highest level of NAIL mRNA.

### EXAMPLE 5

### Tyrosine Phosphorylation of NAIL is Inducible

Four tyrosines are present in the intracellular portion of NAIL. These tyrosines conform to the motif YxxV/I, where x could be any amino acid. This motif resembles the immunoreceptor tyrosine-based activation motif (ITAM) sequences that are present in the cytoplasmic domain of stimulatory receptors such as the T cell receptor (Isakov, Immunol. Res. 16:85-100, 1997) and Fc receptors (Daëron, Ann. Rev. Immunol. 15:203-234, 1997). In these receptors, phosphorylation of the tyrosine residues within the ITAM sequences leads to the rapid recruitment of SH2 domain-containing tyrosine kinases, which participate in the stimulatory signaling cascade. To determine whether NAIL could be tyrosine phosphorylated, CV-1/EBNA cells were transfected with an expression plasmid encoding full-length NAIL. Two days after transfection the cells were incubated with 50 mM Na pervanadate, an inhibitor of protein tyrosine phosphatases, for 5 min. The cells were then lysed in RIPA buffer containing 1% NP-40, 0.5% Na deoxycholate, 50 mM Tris pH 8, 2 mM EDTA, 0.5 mM Na orthovanadate, 5 mM Na fluoride, β-glycerol phosphate and protease inhibitors. The lysates were incubated for 2 hours at 4° C. with 5 μg/ml of anti-phosphotyrosine polyclonal antiserum (Transduction Laboratories, Lexington, Ky.). The immunocomplexes were precipitated by incubation with protein-A Sepharose (Pharmacia, Piscataway, N.J.). After washing, the immunoprecipitates were loaded onto a polyacrylamide gel, electrophoresed under reducing conditions, and transferred to nitrocellulose membranes (Amersham). Western blots were probed with an Ab against NAIL at 2.5 μg/ml and immunocomplexes were detected by enhanced chemiluminescence (NEN, Boston, Mass.). Western blotting revealed that NAIL is not tyrosine phosphorylated in resting cells, but can be rapidly phosphorylated upon incubation of the cells with Na pervanadate (FIG. 2C). Using C1.7 Ab, a single prominent 67 kD band was detected in the lysates from cells treated with Na pervanadate and transfected with full-length NAIL cDNA. This suggests that phosphorylation of NAIL plays a role in its signaling mechanism via the recruitment of specific cytoplasmic signaling molecules.

### EXAMPLE 6

### Preparation of Peripheral Blood Cells

Heparinized peripheral blood from healthy donors was layered over isolymph and centrifuged. PBMC from the resulting interphase were aspirated and washed three times with PBS, resuspended in RPMI1640 medium with 10% FBS and 2 mM glutamine referred to as complete medium (CM) incubated in 37° C. in a humidified incubator with 5% CO₂ and allowed to adhere for 60 min in T175 flasks previously coated with 2% gelatin and precoated with fresh autologous plasma Nonattached lymphocytes (PBL) were gently washed with prewarmed medium and used for generation of NK cells. NK cells were expanded by coculture of PBL with irradiated RPMI-8866 cells as described previously (Perussia et al., Nat. Immun. Cell. Growth Regul. 6:171-188, 1987). At day 8 or 9 of the culture, cells were collected, washed with PBS and depleted of contaminating T cells by magnetic cell separation using magnetic beads coated with anti-CD2 Ab (Miltenyi Inc. Auburn, Calif.) according to the manufacturer's suggestions. Resulting populations of NK cells were always >95% pure as assessed by staining with anti-CD16 and anti-CD56 antibodies. Attached cells were removed by incubation on ice in 20 mM EDTA in CM for 5 min on ice, washed with CM and used for generation of DC as described previously. These cells were usually over 90% CD14⁺ monocytes (Freundlich and Avdalovic, J. Immunol. Methods 62:31-37, 1983). DC were generated after culturing monocytes for 7 d in the presence of GM-CSF (50 ng/ml) and IL-4 (10 ng/ml) as previously described (Sallusto and Lanzavecchia, J. Exp. Med. 179:341-346, 1994). Generated cells were >85% CD1a⁺ as assessed by FACS analysis.

PBB were obtained after removal of sheep RBC-roseting PBMC and positive selection on a magnetic cell separator (Miltenyi Inc.) using anti-CD19 Ab coated magnetic beads according to the manufacturer's suggestion. The resulting B-cell population was always >98% CD20⁺ as determined by flow cytometry.

RPMI-8866, U937, Raji, K562, Daudi, MP-1, and Jurkat cell lines were cultured in suspension in CM and were split twice weekly at a ratio that allowed maintenance of cell concentrations below 10⁶ cells/ml. HepG2, CaCo-2 and T84 were cultured as monolayers in DMEM medium supplemented with 10% FBS, 2 mM glutamine.

### EXAMPLE 7

### Binding of Soluble NAIL-Fc Fusion Protein

Soluble NAIL-Fc (NAIL-Fc) fusion protein was generated and used in FACS analysis in an attempt to identify its potential counterstructure. Flow cytometry was performed as described (Cosman et al., Immunity 7:273-282, 1997). Briefly, cells were incubated with Fc fusion protein in binding buffer for 30 min on ice, washed 3 times in PBS and incubated for an additional 30 min on ice with goat anti human Fcγ PE conjugated Ab (Jackson Immunoresearch Laboratories). After three washes, cells were resuspended in the 3% BSA in PBS (3% PBSA) and analyzed on FACScan (Becton Dickinson). In the blocking experiments titrated concentrations of NAIL-Fc, NAIL-LZ, hCD48-Fc, hCD48-LZ 2B4-Fc or antibodies against human or mouse CD48 or NAIL were used.

Binding of NAIL-Fc could be detected on virtually all subsets of peripheral blood mononuclear cells (PBMC) and several cell lines (FIG. 3A.) Cells of different origin bound NAIL-Fc fusion protein. The highest level of binding could be detected in cell lines of myeloid (U937) and B cell origin (MP-1 and RPMI-8866). Lower levels of NAIL-Fc binding were observed on Jurkat cells and no binding was detected on Daudi or K562 cell lines.

### EXAMPLE 8

### NAIL-CD48 is a Receptor-Ligand Pair

Equilibrium binding assays were performed on transiently expressed hCD48. Full-length hCD48 in the expression vector pDC409 was transfected into CV-1/EBNA cells and these cells were diluted 1:20 into a carrier cell (Daudi). Serial dilutions of NAIL-Fc in binding media (RPMI 1640), 2.5% bovine serum albumin, 20 nM HEPES, 0.02% Na azide [pH 7.2]) were incubated with cells (combination of carrier cells and transiently expressed cells 2.5×10⁶ combined cells/well) for 2 hours at 4° C. in a total of 150 μl/ml in a 96-well microtiter plate. The plate was centrifuged and the supernatants were aspirated. One-hundred fifty microliters of ¹²⁵I goat anti-human IgG Fcγ (Jackson Immunoresearch Laboratories, Inc., West Grove, Pa.; cat #109-006-098) was added to each well and cells were incubated another 1 hour at 4° C. Free and bound probes were separated by the pthalate oil separation method, essentially as described (Dower et al., J.Immunol. 132:751-758, 1984). Goat anti-human IgG Fcγ was labeled with 1211 using solid phase chloramine T analog (Iodogen; Pierce Chemical, Rockford, Ill.) to a specific radioactivity of 2.11×10¹⁵ cpm/mmol. Scatchard analysis revealed high affinity binding of C1.7-Fc to these transfectants (FIG. 3B) with a biphasic curve demonstrating affinities of 1×10⁻¹² and 1.53×10⁻⁹ M. Similarly, high affinity binding (Ka=1.34×10⁹) was detected when ¹²⁵I-labeled hCD48-Fc fusion protein was incubated with CV-1/EBNA cells transfected with full-length C1.7 cDNA (FIG. 3C). No binding of C1.7-Fc or CD48-Fc proteins could be detected on nontransfected cells.

In order to confirm the relevance of our findings on cells transfected with NAIL and CD48 cDNAs, several FACS analyses were performed on NK cells and cell lines. Binding of NAIL-Fc protein to Raji cells was completely blocked when incubation was performed in the presence of a 30-fold molar excess of anti-human CD48 Ab or anti-NAIL Ab NAIL (FIG. 4A). Binding of NAIL-Fc protein to Raji cells was also blocked by soluble CD48, indicating that soluble NAIL polypeptide can bind soluble CD48. The two soluble protein were further shown to bind in co-immunoprecipitation experiments. Binding of NAIL-Fc was also capable of preventing binding of anti-CD48 antibodies to the Raji cell line and of NAIL to NK cells. Titration of reagents used in this experiment suggested comparable affinity of binding of the to relevant soluble Fc fusion proteins and respective MAb. NAIL-Fc fusion protein was also functional as assessed by its ability to inhibit NAIL inducted reverse Ab dependent cell cytotoxicity (rADCC) against P815 targets (FIG. 4B). In this experiment, cytotoxicity of NK cells against the Fc receptor-positive mouse cell line P815 was performed in the presence of anti-NAIL or control anti-CD56 Ab in the presence of different concentrations of NAIL-Fc or control Fc fusion protein. rADCC induced by NAIL Ab was inhibited in the presence of NAIL-Fc fusion protein with 10 μg/ml of this protein being able to almost completely block specific cytotoxicity (FIG. 4B). Addition of NAIL-Fc to the culture had no effect on CD16-mediated rADCC (not shown).

### EXAMPLE 9

### Mouse CD48 is a Ligand for 2B4

Based on the detected homology between NAIL and 2B4, as well as similar biological activities generated in mouse and human NK cells upon stimulation through 2B4 and NAIL, respectively, we next tested if 2B4 would bind to murine CD48 (mCD48). A soluble fusion protein consisting of the extracellular portion of 2B4 and the Fc portion of human IgG1 (2B4-Fc) was generated and used in a binding assay on mouse cells. We tested splenocytes from Balb/C, DBA, CB.17/SCID, C57B1/6, C57B/10, C3H/J strains of mice for binding of 2B4-Fc. Binding of this fusion protein was detected in all tested splenocytes. This binding could be almost completely prevented by the addition of a 20-fold molar excess of specific anti-mCD48 Ab (FIG. 5). Competition for this binding was dose dependent. Both 2B4-Fc and anti-mCD47 Ab were capable of competitively preventing binding of anti-CD48 and 2B4-Fc respectively, to mouse splenocytes. Anti-CD48 Ab had no effect on binding of other Fc fusion proteins capable of staining mouse splenocytes.

### EXAMPLE 10

### NAIL Enhances B Cell Proliferation and Induces Cytokine Production by Dendritic Cells

Costimulation of B cells with anti-CD48 Ab is capable of enhancing Ig secretion, proliferation, and tyrosine phosphorylation of various proteins. These biological effects were observed when crosslinked anti-CD48 Ab was used in combination with cytokines and CD40L stimulation (Klyushnenkova et al., Cell. Immunol. 174:90-98, 1996). In order to determine whether NAIL would have a similar effect in vitro, we purified CD19⁺ peripheral blood B cells (PBB) and stimulated them with immobilized NAIL-Fc in the presence or absence of suboptimal concentrations of IL-4 or CD40L. Proliferation of PBB was determined after culturing cells for 96 hours in a 96-well place at 5×10⁴ cells/well. ³H labeled thymidine (0.5 μCi) in CM was added to the wells for the last 18 hours of culture and cells were harvested onto glass fiber for counting on a gas-phase β-counter (Packard, Meriden, Conn.). In the absence of costimuli, NAIL-Fc had no stimulatory effects on PBB. However, a significant dose-dependent increase in proliferation was observed upon costimulation in the presence of either IL-4 or soluble CD40L (FIG. 6A).

An increase in the secretion of IgM was also observed upon costimulation of human (or mouse) B cells with NAIL-Fc (or 2B4-Fc) in the presence of either IL-4 or soluble CD40L.

To test whether soluble NAIL-leucine zipper (NAIL-LZ) has any biological effect on cells of myeloid origin, we used monocyte-derived DC and incubated them for 48 hours in the presence of various concentrations of NAIL-LZ, control LZ fusion protein, or LPS. NAIL-LZ was capable of inducing production of IL-12p40 and TNFα by DC in a dose-dependent manner (FIG. 6B).

### EXAMPLE 11

### CD48 Upregulates NK Cell Cytotoxicity and Interferon-γ Production

Knowing that CD48 is a natural ligand for NAIL we next tested whether CD48-Fc protein would be capable of exerting biological effects on NK cells similar to those observed using NAIL. Soluble hCD48-Fc (shCD48-Fc) or control Fc were immobilized on 96-well plates precoated with purified goat anti-human Fc Ab. Purified NK cells were added and allowed to settle for 1 hour after which ⁵¹Cr-labeled targets were added at 10⁴ cells/well. Cell targets were labeled with Na₂⁵¹CrO₄ (100 μCi/10⁶ cells) for 1 hour at 37° C. Serial dilutions of effector cells were mixed with targets (10⁴ cells/well) in 96-well round-bottom plates. Cell-free supernatants were harvested after 3-4 hours incubation at 37° C., 5% CO₂, using a cell harvester (Scatron, Sterling, Va.). The percent specific lysis was calculated as ([experimental release cpm−spontaneous release cmp]/[total release cpm−spontaneous release cpm])×100. A significant increase of target lysis by NK cells stimulated with immobilized CD48 protein was noticed (FIG. 7A). This enhancement could be observed when non-activated (FIG. 7A), IL-15 or IFNα activated NK cells were used as effectors (data now shown).

Cytokine levels in cell-free supernatants were determined by double determinant radioimmunoassay (RIA) or ELISA using the following pair of antibodies: B133.1 and 133.5 (for IFNγ), B154.7 and B154.9 (for TNFα), and C11.79 and C8.6 (for IL-12p40) as described (Kubin et al., J. Exp. Med. 180:211-222, 1994). Mouse Mab directed against anti-human molecules were purchased from Immunotech (CD-1a, CD-3, CD-48, NAIL; Miami, Fla.) or Pharmingen (CD-14, CD-16, CD-19, CD-56; San Diego, Calif.). Anti-mouse CD48 Ab HM48-1 was purchased from (Immunotech), BCM-1 from Pharmingen. Goat anti-human IgG Fcγ was purchased from Jackson Immunoresearch Laboratories, mouse anti-human IgG from Zymed (San Francisco, Calif.). Human recombinant cytokines used for stimulation or as standards for RIA/ELISA were purchased from R&D Systems (IL-12 p70 and TNFα; Minneapolis, Minn.), or from Genzyme (IL-10, IFN-γ, IFN-α; Cambridge, Mass.). Human recombinant IL-4, GM-CSF, IL-15, and CD40-L-LZ were produced at Immunex. Enhanced production of IFNγ by NK cells treated with immobilized CD48-Fc protein was also detected. Stimulation of NK cells with immobilized CD48-Fc protein alone did not induce IFNγ production (FIG. 7B). However, the NAIL-CD48 interaction is a potent costimulator in the presence of cytokines such as IL-2, IL-12, or IL-15.

1-47. (canceled)

48. An isolated nucleic acid molecule comprising a polynucleotide encoding a polypeptide at least 80% identical to amino acids 22-221 of SEQ ID NO:2, wherein the polypeptide binds CD48.

49. An isolated nucleic acid molecule of claim 48, wherein the polypeptide acid sequence is at least 90% identical to amino acids 22-221 of SEQ ID NO:2, wherein the polypeptide binds CD48.

50. The isolated nucleic acid molecule of claim 48, wherein the polypeptide comprises amino acids 22-221 of SEQ. ID NO:2.

51. The isolated nucleic acid molecule of claim 48, wherein the polypeptide comprises amino acids 1-221 of SEQ ID NO:2.

52. The isolated nucleic acid molecule of claim 48, wherein the polypeptide comprises amino acids 19-221 of SEQ ID NO:2.

53. The isolated nucleic acid molecule of claim 48, wherein the polypeptide comprises amino acids 19-224 of SEQ ID NO:2.

54. The isolated nucleic acid molecule of claim 48, wherein the polypeptide comprises SEQ ID NO:6.

55. The isolated nucleic acid molecule of claim 48, wherein the polypeptide comprises SEQ ID NO:7.

56. The isolated nucleic acid molecule of claim 48, wherein the polypeptide comprises SEQ ID NO:8.

57. A recombinant vector comprising the nucleic acid molecule of claim 48.

58. A host cell transfected or transduced with the vector of claim 57.

59. An isolated nucleic acid molecule comprising a polynucleotide at least 80% identical to SEQ ID NO:1.

60. A method for the production of NK cell Activation Ligand (NAIL) polypeptide comprising culturing a host cell that has been genetically engineered to express the nucleic acid of claim 48 under conditions promoting expression of the polypeptide.

61. The method of claim 60, further comprising recovering the polypeptide.

62. The method of claim 61, wherein the host cell is a mammalian cell.

63. The method of claim 62, wherein the host cell is a CV-1/EBNA cell.

64. An isolated nucleic acid molecule comprising a polynucleotide selected from the group consisting of:
(a) a nucleic acid molecule having the sequence of SEQ ID NO:
(b) a nucleic acid molecule encoding an amino acid sequence comprising the sequence of SEQ ID NO:2;
(c) a nucleic acid molecule that hybridizes to either strand of a denatured, doublestranded DNA comprising the nucleic acid sequence of (a) or (b) under conditions of moderate stringency in 50% formamide and 6×SSC, at 42° C. with washing conditions of 60° C., 0.5×SSC, 0.1% SDS, wherein said nucleic acid sequence encodes an amino acid sequence having at least 80% identity with SEQ ID NO:2; and
(d) a fragment of any one sequence of (a)-(c) comprising at least 25 contiguous nucleotides.

65. An isolated nucleic acid molecule comprising a polynucleotide that encodes a polypeptide having an amino acid sequence selected from the group consisting of:
(a) amino acids 22-221 of SEQ ID NO:2;
(b) amino acids 1-221 of SEQ ID NO:2;
(c) amino acids 246-365 of SEQ ID NO:2;
(d) amino acids 19-221 of SEQ ID NO:2;
(e) amino acids x-y of SEQ ID NO:2, wherein x is an integer selected from the group consisting of 19 through 22, inclusive, and y is an integer selected from the group consisting of 221 through 224, inclusive;
(f) SEQ ID NO:7; and
(g) SEQ ID NO:8.

66. A recombinant vector that directs the expression of the nucleic acid molecule of claim 65.

67. An isolated polypeptide comprising an amino acid sequence selected from the group consisting of:
(a) an amino acid sequence encoded by a nucleic acid molecule of claim 1;
(b) amino acids 22-221 of SEQ ID NO:2;
(c) amino acids 1-221 of SEQ ID NO:2;
(d) amino acids 246-365 of SEQ ID NO:2;
(e) amino acids 19-221 of SEQ ID NO:2;
(f) amino acids x-y of SEQ ID NO:2, wherein x is an integer selected from the group consisting of 19 through 22, inclusive, and y is an integer selected from the group consisting of 221 through 224, inclusive;
(g) SEQ ID NO:7; and
(h) SEQ ID NO:8.

68. An isolated antibody that binds to a polypeptide consisting of amino acids 1-365 of SEQ ID NO:2, wherein said antibody binds to an epitope other than that bound by C1.7 mAb.

69. The isolated antibody according to claim 68, wherein the antibody is a monoclonal antibody.

70. A host cell transfected or transduced with the vector of claim 67.

71. A method for the production of NAIL polypeptide comprising culturing a host cell that has been genetically engineered to express a human NAIL polypeptide under conditions promoting expression.

72. The method of claim 71, further comprising recovering the polypeptide.

73. The method of claim 71, wherein the host cell is a mammalian cell.

74. An immunogenic composition comprising a recombinant or synthetic human NAIL polypeptide and a physiologically acceptable diluent.

75. An isolated DNA fragment of the nucleic acid molecule of SEQ ID NO:1, wherein said fragment encodes a polypeptide that binds CD48, stimulates cell activation through CD48, or inhibits cell activation through NAIL.

76. A polypeptide encoded by the DNA fragment of claim 75.

77. An oligomer comprising at least two monomers of the polypeptide of claim 75.

78. The polypeptide of claim 76, fused to a heterologous polypeptide.

79. A method for detecting CD48 comprising exposing biological material comprising CD48 to a NAIL polypeptide and detecting complexes formed between NAIL polypeptide and CD48.

80. A method for chelating CD48 comprising exposing biological material comprising CD48 to a soluble NAIL polypeptide, whereby CD48 is chelated.

81. A method for inhibiting binding of CD48 to NAIL polypeptide on a cell surface comprising exposing a biological material comprising CD48 and a cell comprising NAIL on the cell surface to a soluble NAIL polypeptide, whereby binding of CD48 to NAIL polypeptide on the cell surface is inhibited.

82. A method of screening for inhibitors of the binding of CD48 to NAIL polypeptide comprising:
(A) exposing a NAIL polypeptide to a CD48 polypeptide in the presence of a test sample;
(B) comparing the level of complexes formed to a level formed in a control sample in the absence of said test compound, wherein a lower level of complexes in the presence of said test sample is indicative of the presence of an inhibitor in said test sample.

83. The method of claim 82, wherein said method is a yeast two-hybrid assay.

84. A method of stimulating B cells comprising exposing a B cell expressing CD48 to a soluble NAIL polypeptide, whereby said B cell is stimulated, wherein said B cell is optionally activated with IL-4, IL-10, or CD40L.

85. The method of claim 84, wherein an immunogen or vaccine is incubated with said cell.

86. A method for stimulating NK cells or cytotoxic T cells comprising exposing an NK cell expressing NAIL polypeptide or a cytotoxic T cell expressing NAIL polypeptide to soluble CD48 polypeptide, whereby said cell is stimulated.

87. A method of inhibiting the proliferation of cancer cells comprising exposing a cancer cell expressing CD48 to a soluble NAIL polypeptide, whereby the proliferation of said cancer cell is inhibited.

88. A method comprising administering a soluble NAIL polypeptide to an individual in an amount sufficient to stimulate B cells in the individual, increase secretion of IgM by B cells in the individual, stimulate dendritic cells in the individual and/or increase production of TNFcz and/or IL-12 by dendritic cells in the individual.
